(19) United States
(12) Patent Application Publication
(10) Pub. No.: US 2023/0028754 A1
(43) Pub. Date: Jan. 26, 2023

(54) MICROFLUIDIC CARTRIDGES FOR PROCESSING PARTICLES AND CELLS

(71) Applicant: GPB Scientific, Inc., Richmond, VA (US)

(72) Inventors: Alison SKELLEY, Richmond, VA (US); Yasna BEHMARDI, Richmond, VA (US); Anthony WARD, Richmond, VA (US); Khushroo GANDHI, Richmond, VA (US)

(21) Appl. No.: 17/788,697

(22) PCT Filed: Dec. 23, 2020
(86) PCT No.: PCT/US2020/066812

§ 371 Date: Jun. 23, 2022

Related U.S. Application Data
(60) Provisional application No. 62/954,478, filed on Dec. 28, 2019.

(51) Int. Cl. B01L 3/00

(52) U.S. Cl. CPC B01L 3/502761; B01L 2200/0652; B01L 2200/04; B01L 2300/0887; B01L 2300/0864; B01L 2300/0851

(57) ABSTRACT

Described herein is a microfluidic cartridge for purifying target particles or target cells of a predetermined size from contaminants in a sample, the cartridge comprising a first and a second planar support the first and second planar support each having a top surface and a bottom surface, wherein the top surface of the first and/or second planar support comprises at least one embedded channel extending from one or more inlets to one or more outlets; the at least one embedded channel comprising a plurality of obstacles, wherein the microfluidic cartridge comprises at least one void space configured to be deformed when assembling the first and second planar supports into the microfluidic cartridge.

### CROSS-REFERERENCE TO RELATED APPLICATIONS

This Application claims the benefit of U.S. Provisional Application Ser. No. 62/954,478 filed on Dec. 28, 2019, the entirety of which is incorporated by reference herein.

### BACKGROUND

The preparation of cells for personalized therapy often requires the collection of biological material from a patient, the purification of a specific cell type from the material collected and the engineering or growth of the purified cells. In the case of CAR T cell therapy, a large volume of blood, or a blood derived apheresis or leukapheresis preparation, will typically need to be processed to obtain a T cell preparation suitable for genetic engineering and expansion. Microfluidic size-based procedures offer a processing option that is rapid, gentle and versatile. However, there are factors, including the deposit of biological debris during the operation of microfluidic devices, that can slow processing and lead to poorer purifications. Thus, the development of better performing devices and better methods for increasing the rate at which biological materials can be purified are of considerable interest.

### SUMMARY

Described herein are certain separation cartridges for use with microfluidic deices with improvement to allow manufacture of cartridges with delicate features such as posts or obstacles for size-based separation, holding pens for cells, and other microfluidic features. Also described are certain separation cartridges for use with microfluidic deices with improvement to allow for fluid flow in a cartridge that has multiple lanes or channels, such as separator walls that extend for certain lengths to prevent unwanted mixing, turbulent flow due to unwanted mixing, and the pulsed nature of the delivery attributed to some positive displacement pumps.

Described herein in one aspect is a microfluidic cartridge for purifying target particles or target cells of a predetermined size from contaminants in a sample, the cartridge comprising a first and a second planar support the first and second planar support each having a top surface and a bottom surface, wherein the top surface of the first and/or second planar support comprises at least one embedded channel extending from one or more inlets to one or more outlets; the at least one embedded channel comprising a plurality of obstacles.

In certain embodiments, the microfluidic cartridge comprises at least one void space configured to be deformed when assembling the first and second planar supports into the microfluidic cartridge. In certain embodiments, the bottom surface of the first and second planar support comprise at least one void space configured to be deformed when the bottom of the first planar support is pressed to the bottom of the second planar support. In certain embodiments, the at least one void space is configured to prevent damage, displacement, or deformation of the at least one embedded channel, the one or more inlets, the one or more outlets, the plurality of obstacles, or a combination thereof. In certain embodiments, the at least one void space is configured to prevent damage, displacement, or deformation of the plurality of obstacles. In certain embodiments, the microfluidic cartridge comprises a 1:1 ratio of void spaces to channels. In certain embodiments, the at least one void space comprises a total surface area that is at least about 90% of a total surface area of the at least one embedded of channel. In certain embodiments, the at least one void space comprises a total surface area that is at least about 100% of a total surface area of the at least one embedded channel. In certain embodiments, the at least one void space comprises a total surface area that is at least about 110% of a total surface area of the at least one embedded channel. In certain embodiments, the at least one void space is separated into two or more void spaces positioned on the bottom surface of the first and/or second planar support opposite the array of obstacles. In certain embodiments, the planar support is fabricated from two layers of material bonded together. In certain embodiments, the microfluidic cartridge further comprises an obstacle bonding layer that is bonded to a surface of the planar support and bonded to a top surface of the plurality of obstacles in the at least one embedded channel to prevent fluid or sample from flowing over the plurality of obstacles during operation of the cartridge. In certain embodiments, the obstacle bonding layer comprises one or more passages fluidically connected to the one or more inlets of the at least one embedded channel which permits the flow of sample into the at least one embedded channel and one or more passages fluidically connected to the one or more outlets of the at least one embedded channel that permits the flow of fluid out from the one or more outlets. In certain embodiments, the obstacles are positioned so as to define a critical size of the cartridge such that, when a sample is applied to an inlet of the cartridge and flows to an outlet, particles or cells in the sample larger than the critical size are separated from particles or cells in the sample smaller than the critical size. In certain embodiments, the one or more outlets comprise at least one product outlet, wherein the target particles or target cells that have a size larger than the critical size of the cartridge are directed to the at least one product outlet. In certain embodiments, the one or more outlets comprise at least one waste outlet, and the contaminants that have a size smaller than the critical size of the cartridge flow to the at least one waste outlet. In certain embodiments, the plurality of obstacles have a diamond or elongated diamond shape. In certain embodiments, the plurality of obstacles have a circular or ellipsoid shape. In certain embodiments, the plurality of obstacles have a hexagonal shape. In certain embodiments, the plurality of obstacles are elongated perpendicularly to the direction of fluid flow such that they have a horizontal length (P1) that is different from their vertical length (P2). In certain embodiments, P1 is about 10 μm to about 160 μm and P2 is about 5 μm to about 80 μm. In certain embodiments, P1 is about 10 μm to about 80 μm and P2 is about 15 μm to about 60 μm. In certain embodiments, P1 is about 15 μm to about 30 μm and P2 is about 25 μm to about 45 μm. In certain embodiments, P1 is about 40 μm and P2 is about 20 μm. In certain embodiments, P1 is 50 to 150% longer than P2. In certain embodiments, the plurality of obstacles have vertices that extend into parallel gaps such that the gaps are flanked on either side by one or more vertices pointing toward one another but not directly opposite one another. In certain embodiments, the plurality of obstacles have vertices that extend into perpendicular gaps such that the gaps are flanked on either side by vertices pointing toward one another and that are directly opposite one another. In certain embodiments, the plurality of obstacles is arranged into at least at least 1 column. In certain embodiments, the plurality of obstacles is arranged into at least at least 10 columns. In certain embodiments, the plurality of obstacles is arranged into at least at least 30 columns. In certain embodiments, the plurality of obstacles is arranged into at least 50 columns. In certain embodiments, the plurality of obstacles is arranged into at least about 60 columns. In certain embodiments, the plurality of obstacles is arranged into at least at least about 50 rows. In certain embodiments, the plurality of obstacles is arranged into at least at least about 100 rows. In certain embodiments, the plurality of obstacles is arranged into at least at least about 300 rows. In certain embodiments, the plurality of obstacles is arranged into at least at least about 600 rows. In certain embodiments, the first or second planar support comprise at least 10 embedded channels. In certain embodiments, the first and/or second planar support comprise at least 20 embedded channels. In certain embodiments, the first and/or second planar support comprise about 28 embedded channels. In certain embodiments, the first and/or second planar support comprise about 30 embedded channels. In certain embodiments, the first and/or second planar support comprise at least about 50 embedded channels. In certain embodiments, the one or more inlets of the microfluidic cartridge are comprised of at least one or more sample inlets and at least one or more fluid inlets; wherein the at least one or more sample inlets are separated from the at least one or more fluid inlets by a separator wall that extends from the one or more sample inlets into the array of obstacles in the at least one embedded channel toward the outlets and that is oriented parallel to the direction of fluid flow. In certain embodiments, the separator wall extends for at least 10% of the length of the plurality of obstacles. In certain embodiments, the separator wall extends for at least 20% of the length plurality of obstacles. In certain embodiments, the separator wall extends for at least 60% of the length plurality of obstacles. In certain embodiments, the one or more inlets, the one or more outlets, or both, are fluidically connected to a first peristaltic pump, a second peristaltic pump, or both. In certain embodiments, the first peristaltic pump and the second peristaltic pump are fluidically connected in serial. In certain embodiments, the first peristaltic pump and the second peristaltic pump are fluidically connected in parallel. In certain embodiments, the cartridge is fabricated from a polymer. In certain embodiments, the polymer is a thermoplastic polymer. In certain embodiments, the thermoplastic polymer is chosen from the group comprising of high-density polyethylene, polypropylene, polyethylene terephthalate, polycarbonate, or cyclic olefin copolymer. In certain embodiments, the thermoplastic polymer is cyclic olefin copolymer.

Described herein in one aspect is a microfluidic cartridge for purifying target particles or target cells of a predetermined size from contaminants in a sample, the cartridge comprising a first and a second planar support the first and second planar support each having a top surface and a bottom surface, wherein the top surface of the first and/or second planar support comprises at least one embedded channel extending from one or more inlets to one or more outlets; the at least one embedded channel comprising a plurality of obstacles, wherein the microfluidic cartridge comprises at least one void space configured to be deformed when assembling the first and second planar supports into the microfluidic cartridge. In certain embodiments, the bottom surface of the first and second planar support comprise at least one void space configured to be deformed when the bottom of the first planar support is pressed to the bottom of the second planar support. In certain embodiments, the at least one void space is configured to prevent damage, displacement, or deformation of the at least one embedded channel, the one or more inlets, the one or more outlets, the plurality of obstacles, or a combination thereof. In certain embodiments, the at least one void space is configured to prevent damage, displacement, or deformation of the plurality of obstacles. In certain embodiments, the microfluidic cartridge comprises a 1:1 ratio of void spaces to channels. In certain embodiments, the at least one void space comprises a total surface area that is at least about 90% of a total surface area of the at least one embedded of channel. In certain embodiments, the at least one void space comprises a total surface area that is at least about 100% of a total surface area of the at least one embedded channel. In certain embodiments, the at least one void space comprises a total surface area that is at least about 110% of a total surface area of the at least one embedded channel. In certain embodiments, the at least one void space is separated into two or more void spaces positioned on the bottom surface of the first and/or second planar support opposite the array of obstacles. In certain embodiments, the planar support is fabricated from two layers of material bonded together. In certain embodiments, the microfluidic cartridge further comprises an obstacle bonding layer that is bonded to a surface of the planar support and bonded to a top surface of the plurality of obstacles in the at least one embedded channel to prevent fluid or sample from flowing over the plurality of obstacles during operation of the cartridge. In certain embodiments, the obstacle bonding layer comprises one or more passages fluidically connected to the one or more inlets of the at least one embedded channel which permits the flow of sample into the at least one embedded channel and one or more passages fluidically connected to the one or more outlets of the at least one embedded channel that permits the flow of fluid out from the one or more outlets. In certain embodiments, the obstacles are positioned so as to define a critical size of the cartridge such that, when a sample is applied to an inlet of the cartridge and flows to an outlet, particles or cells in the sample larger than the critical size are separated from particles or cells in the sample smaller than the critical size. In certain embodiments, the one or more outlets comprise at least one product outlet, wherein the target particles or target cells that have a size larger than the critical size of the cartridge are directed to the at least one product outlet. In certain embodiments, the one or more outlets comprise at least one waste outlet, and the contaminants that have a size smaller than the critical size of the cartridge flow to the at least one waste outlet. In certain embodiments, the plurality of obstacles have a diamond or elongated diamond shape. In certain embodiments, the plurality of obstacles have a circular or ellipsoid shape. In certain embodiments, the plurality of obstacles have a hexagonal shape. In certain embodiments, the plurality of obstacles are elongated perpendicularly to the direction of fluid flow such that they have a horizontal length (P1) that is different from their vertical length (P2). In certain embodiments, P1 is about 10 μm to about 160 μm and P2 is about 5 μm to about 80 μm. In certain embodiments, P1 is about 10 μm to about 80 μm and P2 is about 15 μm to about 60 μm. In certain embodiments, P1 is about 15 μm to about 30 μm and P2 is about 25 μm to about 45 μm. In certain embodiments, P1 is about 40 μm and P2 is about 20 μm. In certain embodiments, P1 is 50 to 150% longer than P2. In certain embodiments, the plurality of obstacles have vertices that extend into parallel gaps such that the gaps are flanked on either side by one or more vertices pointing toward one another but not directly opposite one another. In certain embodiments, the plurality of obstacles have vertices that extend into perpendicular gaps such that the gaps are flanked on either side by vertices pointing toward one another and that are directly opposite one another. In certain embodiments, the plurality of obstacles is arranged into at least at least 1 column. In certain embodiments, the plurality of obstacles is arranged into at least at least 10 columns. In certain embodiments, the plurality of obstacles is arranged into at least at least 30 columns. In certain embodiments, the plurality of obstacles is arranged into at least 50 columns. In certain embodiments, the plurality of obstacles is arranged into at least about 60 columns. In certain embodiments, the plurality of obstacles is arranged into at least at least about 50 rows. In certain embodiments, the plurality of obstacles is arranged into at least at least about 100 rows. In certain embodiments, the plurality of obstacles is arranged into at least at least about 300 rows. In certain embodiments, the plurality of obstacles is arranged into at least at least about 600 rows. In certain embodiments, the first or second planar support comprise at least 10 embedded channels. In certain embodiments, the first and/or second planar support comprise at least 20 embedded channels. In certain embodiments, the first and/or second planar support comprise about 28 embedded channels. In certain embodiments, the first and/or second planar support comprise about 30 embedded channels. In certain embodiments, the first and/or second planar support comprise at least about 50 embedded channels. In certain embodiments, the one or more inlets of the microfluidic cartridge are comprised of at least one or more sample inlets and at least one or more fluid inlets; wherein the at least one or more sample inlets are separated from the at least one or more fluid inlets by a separator wall that extends from the one or more sample inlets into the array of obstacles in the at least one embedded channel toward the outlets and that is oriented parallel to the direction of fluid flow. In certain embodiments, the separator wall extends for at least 10% of the length of the plurality of obstacles. In certain embodiments, the separator wall extends for at least 20% of the length plurality of obstacles. In certain embodiments, the separator wall extends for at least 60% of the length plurality of obstacles. In certain embodiments, the one or more inlets, the one or more outlets, or both, are fluidically connected to a first peristaltic pump, a second peristaltic pump, or both. In certain embodiments, the first peristaltic pump and the second peristaltic pump are fluidically connected in serial. In certain embodiments, the first peristaltic pump and the second peristaltic pump are fluidically connected in parallel. In certain embodiments, the cartridge is fabricated from a polymer. In certain embodiments, the polymer is a thermoplastic polymer. In certain embodiments, the thermoplastic polymer is chosen from the group comprising of high-density polyethylene, polypropylene, polyethylene terephthalate, polycarbonate, or cyclic olefin copolymer. In certain embodiments, the thermoplastic polymer is cyclic olefin copolymer.

Also described is a microfluidic assembly comprising a plurality of microfluidic cartridges the plurality of microfluidic cartridges are in fluid connection. In certain embodiments, the microfluidic cartridges are stacked. In certain embodiments, the plurality of microfluidic cartridges is two. In certain embodiments, the microfluidic cartridges are in fluid connection in parallel. In certain embodiments, the microfluidic cartridges are in fluid connection in series.

Also described is a method of manufacturing the microfluidic cartridge, wherein the cartridge is fabricated by pressing the bottoms of the first and the second planar support together such that the array of obstacles are not deformed. In certain embodiments, the at least one embedded channel, obstacles, or both are fabricated by embossing, hot embossing, roll to roll embossing, or injection molding. In certain embodiments, the microfluidic cartridge is UV-light cured during fabrication. Also described herein is a method for enriching target particles or target cells of a predetermined size from contaminants in a sample, the method comprising: (a) obtaining a sample comprising the target particles or target cells and the contaminants; (b) separating the target particles or target cells from the contaminants by: (i) applying the sample to one or more sample inlets on the microfluidic cartridge; (ii) flowing the sample to the outlets on the cartridge; and (iii) obtaining a product enriched in target particles or target cells from one or more or outlets while removing the contaminants. In certain embodiments, the target particles or target cells have a size larger than a critical size of the array of obstacles and at least some contaminants have sizes smaller than the critical size of the array of obstacles and wherein target cells or target particles flow to the one or more product outlets where a product enriched in target cells or target particles is obtained and contaminants with a size smaller than the critical size of the array of obstacles flow to one more waste outlets. In certain embodiments, the flow rate of the cartridge is about 400 mL per hour. In certain embodiments, the flow rate of the cartridge is at least about 100 mL per hour or greater. In certain embodiments, the flow rate of the cartridge is at least about 300 mL per hour or greater. In certain embodiments, the flow rate of the cartridge is about 1000 mL per hour. In certain embodiments, the internal pressure of the cartridge is at least about 1.5 pounds per square inch or greater. In certain embodiments, the internal pressure of the cartridge is about 15 pounds per square inch. In certain embodiments, the internal pressure of the cartridge is about 50 pounds per square inch or less. In certain embodiments, the internal pressure of the cartridge is from about 10 pounds per square inch to about 20 pounds per square inch. In certain embodiments, the sample is blood or a blood related product. In certain embodiments, the sample is an apheresis or leukapheresis sample. In certain embodiments, the sample comprises platelets as contaminants. In certain embodiments, the method results in the removal of at least 80% of the platelets from the sample. In certain embodiments, the method results in the removal of at least 90% of the platelets from the sample. In certain embodiments, the method results in the removal of at least 95% of the platelets from the sample. In certain embodiments, the enriched target cells comprise leukocytes. In certain embodiments, the enriched target cells comprise stem cells. In certain embodiments, the enriched target cells comprise peripheral blood mononuclear cells. In certain embodiments, the peripheral blood mononuclear cells comprise CD3+ cells. In certain embodiments, the method further comprises genetically engineering the enriched target cells, to obtain genetically engineered target cells. In certain embodiments, said genetic engineering comprises transfecting or transducing the target cells with a recombinant nucleic acid. In certain embodiments, the enriched target cells or genetically engineered target cells are expanded by culturing them in vitro.

In another aspect described herein is a method of producing chimeric antigen receptor (CAR) T cells, comprising: (a) obtaining a sample comprising T cells; (b) separating the T cells from contaminants by: (i) applying the sample to one or more sample inlets on the microfluidic cartridge; (ii) flowing the sample to the outlets of the cartridge; and (iii) obtaining a product enriched in T cells from the product outlet; (c) genetically engineering the T cells in the enriched product obtained in step b) to produce the chimeric antigen receptors (CARs) on their surface. In certain embodiments, the sample is blood, an apheresis product or a leukapheresis product. In certain embodiments, said genetically engineering the T cells comprises transfecting or transducing the target cells and the genetically engineered target cells are expanded further by growing the cells in vitro.

In another aspect described herein is a method of producing chimeric antigen receptor (CAR) natural killer cells, comprising: (a) obtaining a sample comprising natural killer cells; (b) separating the natural killer cells from contaminants by: (i) applying the sample to one or more sample inlets on the microfluidic cartridge; (ii) flowing the sample to the outlets of the cartridge; and (iii) obtaining a product enriched in natural killer cells from the product outlet; (c) genetically engineering the natural killer cells in the enriched product obtained in step b) to produce the chimeric antigen receptors (CARs) on their surface. In certain embodiments, the sample is a blood sample, an apheresis product, or a leukapheresis product. In certain embodiments, said genetically engineering the natural killer cells comprises transfecting or transducing the target cells and the genetically engineered target cells are expanded further by growing the cells in vitro.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Furthermore, each of U.S. Pat. Nos. 5,427,663; 5,837,115; 6,685,841; 6,913,697; 7,150,812; 7,276,170; 7,318,902; 7,472,794; 7,735,652 US 7,988,840; 8,021,614; 8,282,799; 8,304,230; 8,579,117; U.S. Ser. No. 10/324,011; US 2005/0282293; US 2006/0134599; US 2007/0160503; US 2006/0121624; US 2005/0266433; US 2007/0026381; US 2007/0026413; US 2007/0026414; US 2007/0026415; US 2007/0026417; 2007/0059680; US 2007/0059718; US 2007/0059781; US 2007/0059774; US 2007/0099207; US 2007/0196820; US 2006/0223178; US 2008/0124721; US 2008/0090239; US 2008/0113358; US 2014/0342375; US 2016/0139012; US 2019/0071639; and WO2012094642, is incorporated by reference herein in its entirety. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1G illustrate different operating modes of DLD.

FIG. 2 illustrates various uses of channels with an alternative array of obstacles to that shown in FIGS. 1A-1C.

FIGS. 3A-3D illustrate an embodiment of a device comprising an arrangement of 14 parallel channels that could be used in a microfluidic device.

FIGS. 4A-4D illustrate 2 channels. FIGS. 4B-4D illustrate expanded views of sections of the channels.

FIG. 5 is a diagram of a cross-section of a “bump array” device having equilateral triangularly shaped obstacles disposed in a microfluidic channel.

FIGS. 6A-6B illustrate arrays of diamond shaped posts.

FIGS. 7A-7C depict a stacked separation assembly in which two microfluidic devices are combined into a single unit.

FIGS. 8A-8B depict two channels that might be found in a device depicted in FIG. 7. An expanded view of a section of the channels is shown in FIG. 8B. In this example, the channel has an array of asymmetrically spaced diamond obstacles, in which G1 is larger than G2. The diamonds are offset so each successive row is shifted laterally relative to the previous row.

FIG. 9 shows a stacked assembly of microfluidic devices inside a casing which together may be referred to as a “cassette.”

FIGS. 10A and 10B show a channel bounded by two walls, with a sample inlet and a fluid inlet.

FIG. 11 is a comparison of normalized velocity flow between two equilateral triangular posts (left panel) and normalized velocity flow between two circular posts (right panel).

FIG. 12 is a graph of predicted critical diameter versus the array tilt angle (c) for arrays of triangular (lower line) and circular (upper line) obstacles.

FIG. 13 is a graph illustrating the effect of the tilt angle (“Array Tilt” in the figure) on gap length G.

FIG. 14 is a graph illustrating the effect of obstacle edge roundness (expressed as r/S) on the critical size exhibited on the side of a gap bounded by the edge.

FIG. 15 is a graph illustrating the effect of applied pressure on particle velocity in bump arrays having triangular posts (data shown as triangles) and bump arrays having circular posts (data shown as circles).

FIGS. 16A and 16B: show a cross-sectional view of a single cartridge DLD element comprising 6 layers: 2 layers of DLD microposts, 2 layers of voids space crumple zones for fluidics feeder channels, and 2 end layers. FIG. 16B shows a top view of a non-limiting example DLD layer consisting of an array of elongated diamond or hexagonal posts.

FIGS. 17A-C show a top view of a photograph of a 2 DLD element cartridge loaded into the device cassette. FIG. 17B shows a left-side and top-down view of the DLD cartridge loaded into the device cassette. FIG. 17C shows a right-side top-down view of the DLD cartridge loaded into the device cassette.

FIGS. 18A and B show a specific embodiment for the arrangement of a void space showing a view of the bottom of a planar support (18A) and a cross-sectional view (18B).

FIGS. 19A and B show alternative embodiments for a void space when planar supports are stacked to form a microfluidic cartridge (cross-sectional view shown).

### DETAILED DESCRIPTION

The present invention is primarily concerned with size based microfluidic separations, and especially with the use of DLD in preparing cells that are of therapeutic value. The text herein provides guidance regarding the making and use of microfluidic devices and the use of DLD for carrying out separations involving biological materials.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Definitions

Apheresis: As used herein this term refers to a procedure in which blood from a patient or donor is separated into its components, e.g., white blood cells, platelets and red blood cells. An “apheresis sample” is the product that is the end result of this procedure. More specific terms are “plateletpheresis” (referring to the separation of platelets) and “leukapheresis” (referring to the separation of leukocytes). In this context, the term “separation” refers to the obtaining of a product that is enriched in a particular component compared to whole blood or other starting material and does not mean that absolute purity has been attained.

CAR T cells: The term “CAR” is an acronym for “chimeric antigen receptor.” A “CAR T cell” is therefore a T cell that has been genetically engineered to express a chimeric receptor.

CAR T cell therapy: This term refers to any procedure in which a disease or condition is treated with CAR T cells. Diseases that may be treated include hematological and solid tumor cancers, autoimmune diseases and infectious diseases.

Carrier: As used herein, the term “carrier” refers an agent, e.g., a bead or particle, made of either biological or synthetic material that is added to a preparation for the purpose of binding directly or indirectly (i.e., through one or more intermediate cells, particles or compounds) to some or all of the compounds or cells present. Carriers may be made from a variety of different materials, including DEAE-dextran, glass, polystyrene plastic, acrylamide, collagen, and alginate and will typically have a size of 1-1000 μm. They may be coated or uncoated and have surfaces that may be modified to include affinity agents (e.g., antibodies, activators, haptens, aptamers, particles or other compounds) that recognize antigens or other molecules on the surface of cells. The carriers may also be magnetized and they may comprise particles (e.g., Janus or Strawberry-like particles) that confer upon cells or cell complexes non-size related secondary properties. For example, the particles may result in chemical, electrochemical, or magnetic properties that can be used in downstream processes, such as magnetic separation, electroporation, gene transfer, and/or specific analytical chemistry processes. Particles may also cause metabolic changes in cells, activate cells or promote cell division.

Carriers that bind “in a way that promotes DLD separation”: This term, refers to carriers and methods of binding carriers that affect the way that, depending on context, a cell, protein or particle behaves during DLD. Specifically, “binding in a way that promotes DLD separation” means that: a) binding must exhibit specificity for a particular target cell type, protein or particle; and b) binding must result in a complex that provides for an increase in size of the complex relative to the unbound cell, protein or particle. In the case of binding to a target cell, there must be an increase of at least 2 μm (and alternatively at least 20, 50, 100, 200, 500 or 1000% when expressed as a percentage). In cases where therapeutic or other uses require that target cells, proteins or other particles be released from complexes to fulfill their intended use, then the term “in a way that promotes DLD separation” also requires that the complexes permit such release, for example by chemical or enzymatic cleavage, chemical dissolution, digestion, due to competition with other binders, or by physical shearing (e.g., using a pipette to create shear stress) and the freed target cells, proteins or other particles must maintain activity; e.g., therapeutic cells after release from a complex must still maintain the biological activities that make them therapeutically useful.

Carriers may also bind “in a way that complements DLD separation”: This term refers to carriers and methods of binding carriers that change the chemical, electrochemical, or magnetic properties of cells or cell complexes or that change one or more biological activities of cells, regardless of whether they increase size sufficiently to promote DLD separation. Carriers that complement DLD separation do not necessarily bind with specificity to target cells, i.e., they may have to be combined with some other agent that makes them specific or they may simply be added to a cell preparation and be allowed to bind non-specifically. The terms “in a way that complements DLD separation” and “in a way that promotes DLD separation” are not exclusive of one another. Binding may both complement DLD separation and also promote DLD separation. For example, a polysaccharide carrier may have an activator on its surface that increases the rate of cell growth and the binding of one or more of these carriers may also promote DLD separation. Alternatively, binding may just promote DLD separation or just complement DLD separation.

Sample: The term “sample,” as used herein, generally refers to any sample containing or suspected of containing a nucleic acid molecule or cells. For example, a sample can be a biological sample containing one or more nucleic acid molecules or cells. The biological sample can be obtained (e.g., extracted or isolated) from or include blood (e.g., whole blood), plasma, serum, urine, saliva, mucosal excretions, sputum, stool and tears. The sample may contain blood, a blood product (such as a leukapheresis or apheresis product) also containing an anti-coagulant (e.g., EDTA, EGTA, heparin, citrate, ACD-A, or a thrombin inhibitor). The biological sample can be a fluid or tissue sample (e.g., skin sample). In some examples, the sample is obtained from a cell-free bodily fluid, such as whole blood. In some examples, the sample can include circulating tumor cells. In some examples, the sample is an environmental sample (e.g., soil, waste, ambient air and etc.), industrial sample (e.g., samples from any industrial processes), and food samples (e.g., dairy products, vegetable products, and meat products). The sample may be processed prior to loading into the microfluidic device. The sample may suitably be an apheresis product or a leukapheresis product (e.g., leukopak).

Target cells: As used herein “target cells” are the cells that various procedures described herein require or are designed to purify, collect, engineer etc. What the specific cells are will depend on the context in which the term is used. For example, if the objective of a procedure is to isolate a particular kind of stem cell, that cell would be the target cell of the procedure.

Isolate or purify: Unless otherwise indicated, these terms, as used herein, are synonymous and refer to the enrichment of a desired product relative to unwanted material. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in isolating or purifying the target cell.

The terms “obstacle array” are used synonymously herein and describe an ordered array of obstacles that are disposed in a flow channel through which a cell or particle-bearing fluid can be passed. An obstacle array comprises a plurality of obstacles arranged in a column (along the path of fluid flow). Gaps are formed between the obstacles (along the path of the fluid flow) that allows the passage of cells or other particles. Such arrays or columns can be arranged into one or more repeating rows (perpendicular to the path of fluid flow).

As described herein a channel” or “lane” refers to a plurality of obstacles that are arranged into a discreet separation unit, such channels may be bounded on either side by walls such that discreet lanes are separated. Channels may run in parallel from one or more common inputs to one or more common outputs. Channels may be fluidly connected in series.

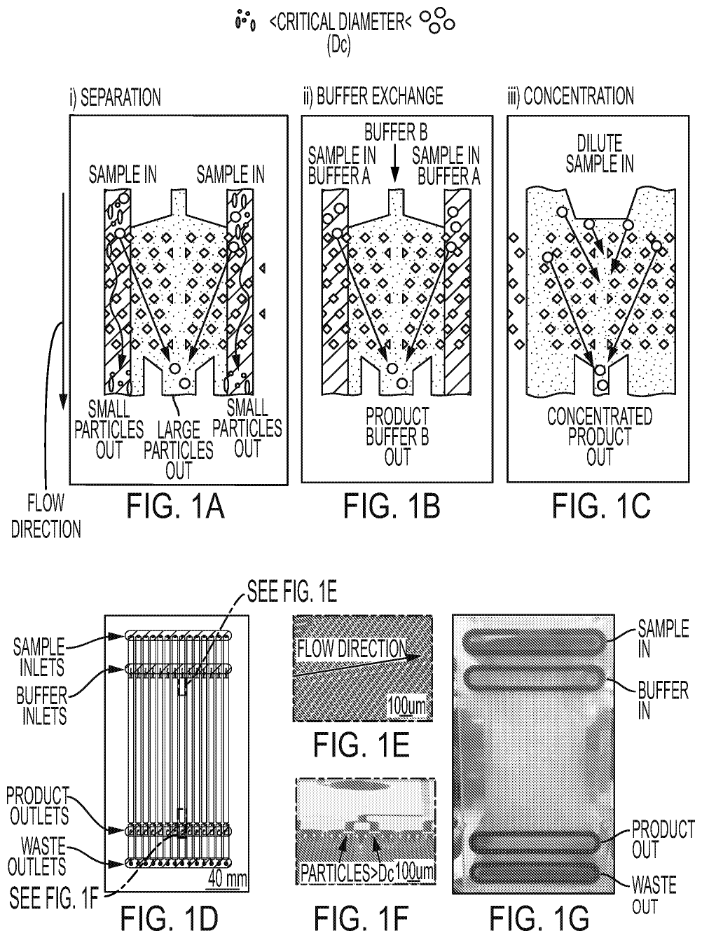

Deterministic Lateral Displacement: As used herein, the term “Deterministic Lateral Displacement” or “DLD” refers to a process in which particles are deflected on a path through a microfluidic obstacle array deterministically, based on their size. This process can be used to separate cells, which is generally the context in which it is discussed herein. However, it is important to recognize that DLD can also be used to concentrate cells and for buffer exchange (see FIG. 1). Processes are generally described herein in terms of continuous flow (DC conditions; i.e., bulk fluid flow in only a single direction). However, DLD can also work under oscillatory flow (AC conditions; i.e., bulk fluid flow alternating between two directions).

Critical size: The “critical size,” “critical diameter” or “predetermined size” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not be displaced.

Fluid flow: The terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in the general direction.

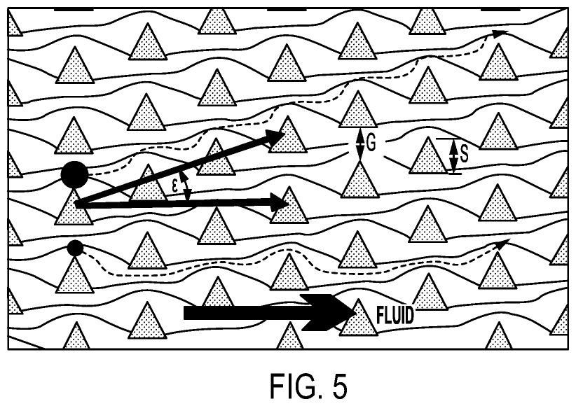

Tilt angle ε: In a bump array device, the tilt angle is the angle between the direction of bulk fluid flow and the direction defined by alignment of rows of sequential obstacles in the array (see FIG. 5).

Array Direction: In an obstacle array device, the “array direction” is a direction defined by the alignment of rows of sequential obstacles in the array. A particle is “deflected” in an obstacle array if, upon passing through a gap and encountering a downstream obstacle, the particle's overall trajectory follows the array direction of the obstacle array (i.e., travels at the tilt angle c relative to bulk fluid flow). A particle is not bumped if its overall trajectory follows the direction of bulk fluid flow under those circumstances.

About: As used herein the term about refers to an amount near a stated amount that is within 10%.

### General Summary

The present invention is concerned with microfluidic devices in which size-based purifications are performed by passing a biological sample through an array of obstacles in a microfluidic channel. It is based, in part, on the concept that by lengthening obstacle gaps perpendicular to the direction of fluid flow and decreasing the length of gaps parallel to fluid flow, cells of a given size can be processed more rapidly.

The device characteristics discussed above can be achieved with a range of obstacle shapes that are oblong, with the most preferred obstacles being diamond or hexagonally shaped. Hexagonally shaped obstacles are most preferred because they provide the same processing advantages as diamonds but result in a device that is easier to manufacture and more resistant to biofouling.

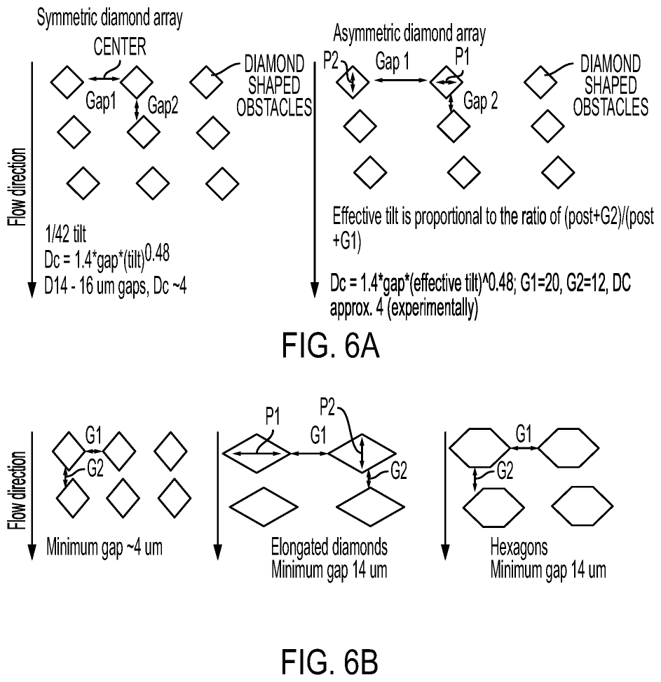

Although using asymmetric gaps improves throughput and allows devices to run longer, the narrowing of parallel gaps can create practical problems for the large-scale production of devices, particularly with respect to embossing, molding or demolding. This problem can be reduced and the need for a narrow gap somewhat offset by using polygonal-shaped, elongated obstacles that, preferably, have vertices pointing toward one another in parallel gaps but where the vertices are offset from one another (as opposed to being directly opposite one another, see FIG. 6A-6B). This design reduces flow through the parallel gaps (also called minor flux) by making the gap longer, not narrower. In contrast, vertices in perpendicular gaps preferably are directly opposite from one another. Thus, a primary characteristic of the devices disclosed herein is the presence of obstacle arrays in which perpendicular gaps and parallel gaps are asymmetric, i.e., they are not the same size. By varying spacing, it is possible to decrease resistance to flow compared to devices that separate particles and cells in the same size range but that have perpendicular and parallel gaps of the same length.

For some samples, biofouling and mixing of fluids as sample is fed onto devices may continue to affect separations. For example, biofouling at the entrance of an array may force a blood or apheresis sample to prematurely spread to a second fluid stream, resulting in platelet and red blood cell contamination in a leukocyte target cell product. A separator wall positioned so as to separate sample inlets from inlets for other fluids and terminating part-way down the channel, may be used to isolate the biofouling area and temporarily prevent contact between flow streams. As a result, the co-flowing fluids have limited time for diffusional mixing and purifications of target cells or particles may be improved. Typically, a separator wall will extend from a sample inlet for a distance of anywhere from 10 to 50% of the length of the microfluidic channel, but a wall may be shorter or longer depending on circumstances associated with a separation.

Another advantage of separator walls is that they reduce unwanted mixing that may occur when a fluctuating pressure source is used to propel a sample and other fluids through a device. For example, a peristaltic pump may be used to drive fluids through a device and has the advantage of maintaining a closed system environment, i.e., sample does not touch the interior of the pump but only travels through tubing which is squeezed by the pump head. However, peristalsis may create regular surges of pressure that tend to cause flow streams to mix. When a separator wall is present, it acts as a baffle for these surges, limiting the unwanted mixing that would otherwise occur. As a result, an improved separation should be realized.

Another characteristic of the present microfluidic devices is that they may be used as part of an assembly in which two or more devices are stacked together and fed through a common manifold. Each stacked microfluidic device comprises a planar support with one or more embedded channels, each containing a separate obstacle array. Supports will typically have multiple channels which, in some instances, may be embedded in both the top and bottom surfaces of a support. Using multiple channels on a device and multiple devices in an assembly allows large volumes of sample to be processed microfluidically. For example, the assemblies of microfluidic devices described herein may be designed to process greater than 100 mL of sample (e.g., an undiluted apheresis sample) per hour with, depending on specific processing objectives, higher volumes (greater than 200, 300, 400 or 500 mL per hour) being preferred.

Overall, the devices of the invention are characterized by some or all of the following: 1) asymmetrically arranged obstacles in which gaps perpendicular to bulk fluid flow are of a different length than gaps parallel to bulk fluid flow; 2) elongated polygonally shaped obstacles with vertices extending into gaps; 3) vertices on either side of parallel gaps that are offset with respect to one another; 4) vertices on either side of perpendicular gaps that are, preferably, directly opposite one another; 5) one or more separator walls segregating sample inlets from inlets for other fluids and that extend part way down channels; 6) the optional use of peristalsis, or other fluctuating pressure sources, to propel sample and other fluids through devices with separator walls; and 7) the assembly of multiple individual microfluidic devices into stacked assemblies with each device having multiple channels.

### Summary of Specific Embodiments

In a first aspect, the invention is directed to a microfluidic device for purifying target particles or target cells of a predetermined size from contaminants in a sample. The device has a planar support that will typically be rectangular and can be made of any material compatible with a separation method, including silicon, glasses, hybrid materials or (preferably) polymers. The support will have a top surface and a bottom surface, one or both of which have at least one embedded channel extending from one or more sample inlets and one or more distinct fluid inlets, to one or more product outlets and one or more distinct waste outlets. Fluid inlets (as opposed to sample inlets) may sometimes be referred to as “buffer” or “wash” inlets and, depending on the objectives of a separation may be used to transport a variety of fluids into channels. Unless otherwise indicated by usage or context, it will be understood that a “fluid” may be a buffer, contain reagents, constitute growth medium for cells or generally be any liquid, and contain any components, compatible with operation of a device and the objectives of the user.

When fluid is applied to a device through a sample or fluid inlet, it flows through the channel toward the outlets, thereby defining a direction of bulk fluid flow. In order to separate cells or particles of different sizes, the channel includes an array of obstacles organized into columns that extend longitudinally along the channel (from inlet to outlet), and rows that extend laterally across the channel. Each subsequent row of obstacles is shifted laterally with respect to the previous row, thereby defining an array direction that deviates from the direction of bulk fluid flow by a tilt angle (ε). The obstacles are positioned so as to define a critical size such that when a sample is applied to an inlet of the device and flows to an outlet, particles or cells in the sample larger than the critical size follow in the array direction and particles smaller than the critical size flow the direction of bulk fluid flow, thereby resulting in a separation.

Adjacent obstacles in a row of the array are separated by a gap, G1, that is perpendicular to the direction of bulk fluid flow and adjacent obstacles in a column are separated by a gap, G2, which is parallel to the direction of bulk fluid flow (see FIGS. 6A and 6B). One characteristic of the present devices is that the ratio of the size of gap G2 to the size of gap G1 does not equal 1, with G1 typically being wider than G2 (e.g., by 10-100%). The obstacles in an array each have at least two vertices and are positioned so that each gap is flanked on either side by at least one vertex. In preferred embodiments, the vertices extend into parallel gaps so that the gaps are flanked on either side by one or more vertices pointing toward one another but not directly opposite one another and/or obstacles have vertices that extend into perpendicular gaps such that the gaps are flanked on either side by vertices pointing toward, and directly opposite to, one another (see FIGS. 6A and 6B).

The microfluidic devices will also typically have an obstacle bonding layer that is bonded to a surface of the planar support and bonded to the obstacles in channels to prevent fluid or sample from flowing over obstacles during operation of the device. This obstacle bonding layer may comprise one or more passages fluidically connected to the inlets of the channel and to the outlets of the channel which permit the flow of fluid.

In general, the microfluidic devices will be used to separate target particles or target cells having a size larger than the critical size of the device from contaminants with sizes smaller than the critical size. When a sample containing the target cells or particles is applied to a device through a sample inlet and fluidically passed through the channel, the target cells or target particles will flow to one or more product outlets where a product enriched in target cells or target particles is obtained. The term “enriched” as used in this context means that the ratio of target cells or particles to contaminants is higher in the product than in the sample. Contaminants with a size smaller than the critical size will flow predominantly to one more waste outlets where they may be either collected or discarded.

Although the objective of a separation will generally be to separate target cells or particles from smaller contaminants, there may be times when a user wants to separate target cells or particles from larger contaminants. In these instances, a microfluidic device may be used with a critical size larger than the target cells or particles but smaller than the contaminants. Combinations of two or more obstacle arrays with different critical sizes, either on a single device or on multiple devices, may also be used in separations. For example, a device may have channels with a first array of obstacles that has a critical size larger than T cells but smaller than granulocytes and monocytes and a second array with a critical size smaller than T cells but larger than platelets and red blood cells. Processing of a blood sample on such a device allows for the collection of a product in which T cells have been separated from granulocytes, monocytes, platelets and red blood cells. The order of the obstacle arrays should not be of major importance to the result, i.e., an array with a smaller critical size could come before or after an array with a larger critical size. Also arrays with different critical sizes can be on separate devices that cells pass through.

Wide arrays and multiple outlets may be used for the collection multiple products, e.g., monocytes may be obtained at one outlet and T cells at a different outlet. Thus, using multiple arrays and multiple outlets may permit the concurrent collection of several products that are more purified than if a single array had been used. As further discussed below, high throughputs may be maintained by using many devices stacked together.

Preferably, the obstacles used in the microfluidic devices have a polygonal shape, with diamond or hexagonally shaped obstacles being preferred. The obstacles will also generally be elongated so that their length perpendicular to bulk fluid flow (P1) is different (generally longer) than their width parallel to bulk fluid flow (P2) by, for example, 10-100% (see FIG. 6B). Typically, P1 will be longer than P2 by at least 15%, 30%, 50%, 100% or 150%. Expressed as a range, P1 may be 10-150% (15-100%; or 20-70%) longer than P2.

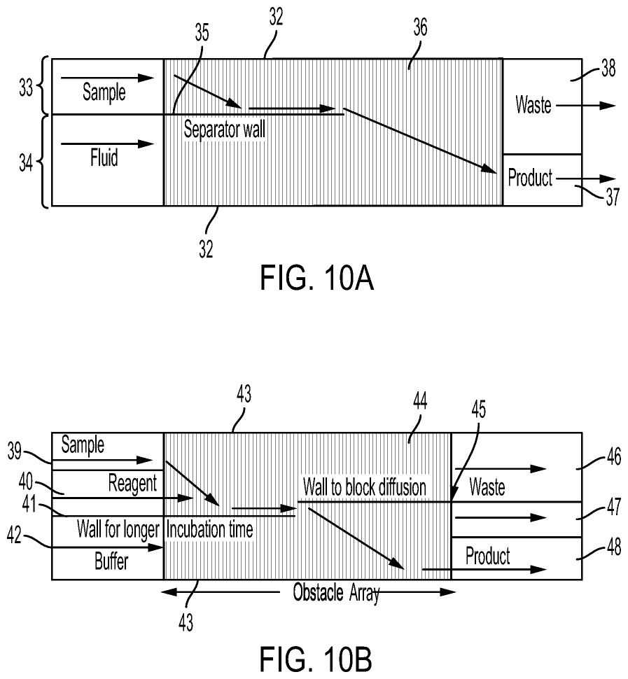

Microfluidic devices may also include a separator wall that extends from the sample inlet of a device, where it separates the sample inlet from fluid inlets and prevents mixing, into the array of obstacles in the channel (see FIGS. 10A and 10B). The separator wall is oriented parallel to the direction of bulk fluid flow and extends toward the sample and fluid outlets. The wall terminates before reaching the end of the channel, allowing sample and fluid streams to contact one another thereafter. It should generally extend at for a distance of at least 10% of the length of the array of obstacles but may extend for at least 20%, 40%, 60%, or 70% of the array. Expressed as a range the wall will typically extend for 10-70% of the length of the array of obstacles. More than one separator wall may also be present in a device and, depending on the objectives of a separation, may be positioned in different ways.

In order to increase the rate at which volume can be processed, a stacked separation assembly can be made by overlaying a first microfluidic with one or more stacked devices, wherein the bottom surface of each stacked device is in contact with either the top surface, or an obstacle bonding layer on the top surface, of the first microfluidic device or with the top surface, or the obstacle bonding layer on the top surface, of another stacked device. Sample is provided to the sample inlets of all devices though a first common manifold and fluid is supplied to the fluid inlets through a second manifold that may or may not be the same as the first manifold. Product is removed from the product outlets through one or more product conduits and waste is removed from the waste outlets through one or more waste conduits that are different from the product conduits. In general, a stacked separation assembly will have 2 to 9 stacked devices together with the first microfluidic device. However, a larger number of devices may also be used. In addition, the top surface of supports, and/or the bottom surface, may have multiple (e.g., 2-40 or 2-30) embedded channels and be used in purifying target particles or target cells.

Stacked separation assemblies may have a reservoir bonding layer which is attached to the bottom surface of the first microfluidic device and/or to the top surface of a stacked microfluidic device. The reservoir bonding layer should include a first end with one or more passages permitting the flow of fluid to inlets on the channels and optionally, one or more passages that permit the flow of fluid to, or from, the product and waste outlets of channels at a second end, opposite to first end and separated by material impermeable to fluid.

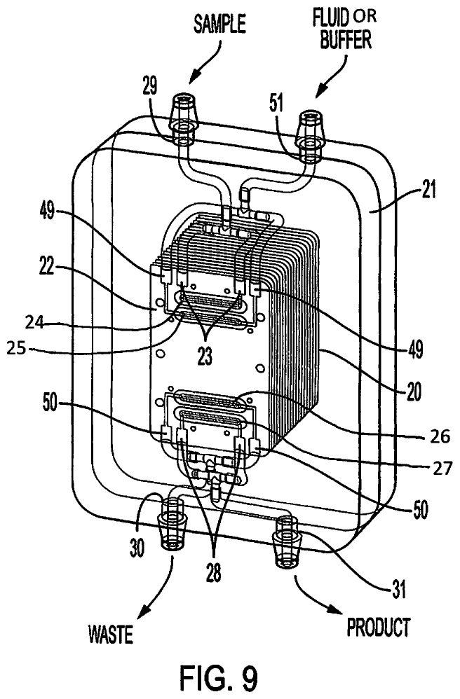

As shown in FIG. 9, stacked assemblies of devices may be supported in a cassette characterized by the presence of an outside casing with ports allowing for the transport of sample and fluids into the cassette and products and waste out of the cassette. The figure shows a cassette with two inlet ports and two outlet ports. However, multiple ports into and out of a cassette may be used and several products may be collected essentially simultaneously. It will also be recognized that cassettes can be part of a system in which there are components that are well known and commonly used in the art. Such common components include, pumps, valves and processors for controlling fluid flow; sensors for monitoring system parameters such a flow rate and pressure; sensors for monitoring fluid characteristics such a pH or salinity; sensors for determining the concentration of cells or particles; and analyzers for determining the types of cells or particles present in the cassette or in material collected from the cassette. More generally, any equipment known in the art and compatible with the cassettes, the material being processed, and the processing objectives may be used.

In another aspect, the invention is directed to a method for purifying target particles or target cells of a predetermined size from contaminants by obtaining a sample comprising the target particles or target cells and contaminants and carrying out a purification using any of the microfluidic devices or stacked separation assembles discussed herein. Purification is accomplished by applying the sample to one or more sample inlets on any of the microfluidic devices discussed above or to sample inlets on the first microfluidic device or a stacked device in an assembly of devices. A manifold may be used to apply sample to inlets, particularly when using stacked devices. Samples are then flowed through the channel to the outlets of devices. Generally, the target particles or target cells will have a size larger than the critical size of the array of obstacles on devices and at least some contaminants will have sizes smaller than the critical size. As a result, the target cells or target particles will flow to one or more product outlets where a product enriched in target cells or target particles is obtained and contaminants with a size smaller than the critical size will flow to one more waste outlets. As noted previously however, there may be instances where the target cells or target particles are smaller than contaminants and devices are chosen with a critical size larger than the target cells or particles and smaller than the contaminants. In these cases, the general operation of devices will be essentially the same but contaminants will flow in the array direction and target cells or particles will proceed in the direction of bulk fluid flow.

The sample may be obtained from an individual or a patient, especially a patient with cancer, an autoimmune disease or an infectious disease. In a certain embodiment, the sample is blood or is derived from blood (e.g., an apheresis or leukapheresis sample), and the target cells are dendritic cells, leukocytes (especially T cells), stem cells, B-cells, NK-cells, monocytes or progenitor cells. The contaminants in these instances will typically include red blood cells and/or platelets. The purification should result in a product enriched in target cells and in which at least 80% (preferably 90% and more preferably 95%) of the platelets and/or red blood cells from the sample have been removed.

Once purified target cells are obtained, they may be genetically engineered, by transfecting or transducing them with recombinant nucleic acids. They may then, optionally, be expanded in culture and, ultimately, be used in treatment of the patient from whom the sample was obtained.

Of particular interest, the invention includes a method for producing chimeric antigen receptor (CAR) T cells, by: a) obtaining a sample comprising T cells; b) separating the T cells from contaminants by applying the sample to one or more sample inlets on any of the microfluidic devices or stacked devices discussed herein; c) flowing the sample to the outlets of the device; and d) obtaining a product enriched in T cells from a product outlet. Once T cells are recovered, they are genetically engineered, preferably by transfecting or transducing them with a recombinant nucleic acid, so that they express chimeric antigen receptors on their surface. The genetically engineered target cells are expanded by growing the cells in vitro and may be administered therapeutically to the patient that provided the sample.

The sample containing T cells is preferably blood, an apheresis product, or a leukapheresis product from a patient with cancer, an autoimmune disease or an infectious disease, or from an HLA matched (to a patient to be treated) donor. The cells may be bound to one or more carriers in a way that promotes or complements DLD separation and cells or complexes may then be purified by DLD. The invention includes the CAR T cells made and CAR T cell therapies in which the CAR T cells are used.

### I. Designing Microfluidic Cartridges

The present disclosure provides microfluidic cartridges (i.e. devices, chips, cassettes, plates, microfluidic devices, cartridges, DLD devices, etc.) for purifying particles or cells. A microfluidic cartridge of the present disclosure may operate using a DLD method. A microfluidic cartridge of the present disclosure may be formed from a polymeric materials (e.g. thermoplastic), and may include one or more of a first planar support having a top surface and a bottom surface, and a second planar support having a top surface and a bottom surface, wherein the top surface of the first and second planar support comprises at least one embedded channel extending from one or more inlets to one or more outlets; the at least one embedded channel comprising an array of obstacles, wherein the bottom surface of the first and second planar support comprises a void space configured to be deformed when a the bottom of the first planar support is pressed to the bottom of the second planar support. A microfluidic cartridge of the present disclosure may be a single-use or disposable device. As an alternative, the microfluidic cartridge may be multi-use device. The use of polymers (e.g., thermoplastics) to form the microfluidic structure may allow for the use of an inexpensive and highly scalable soft embossing process while the void space may provide an improved ability to be manufactured quickly and avoid damage to the obstacles (i.e. posts, DLD arrays, etc.) during the manufacturing process.

The cartridges described herein may operate via deterministic lateral displacement, or DLD. Referring to FIGS. 1A-1G, DLD may include three different operating modes. The operating modes include: i) Separation (FIG. 1A), ii) Buffer Exchange (FIG. 1B) and iii) Concentration (FIG. 1C). In each mode, particles above a critical diameter are deflected in the direction of the array from the point of entry, resulting in size selection, buffer exchange or concentration as a function of the geometry of the device. In all cases, particles below the critical diameter pass directly through the device under laminar flow conditions and subsequently off the device. FIG. 1D shows a 14 lane DLD design used in separation mode. The full length of the separation zone of the microfluidic cartridge may be about 75 mm and the width may be about 40 mm, with each individual channel being about 1.8 mm across. FIGS. 1E-1F are enlarged views of a plastic diamond post array and consolidating collection ports for the exits. FIG. 1G depicts a leukapheresis product being processed using a device at 10 PSI.

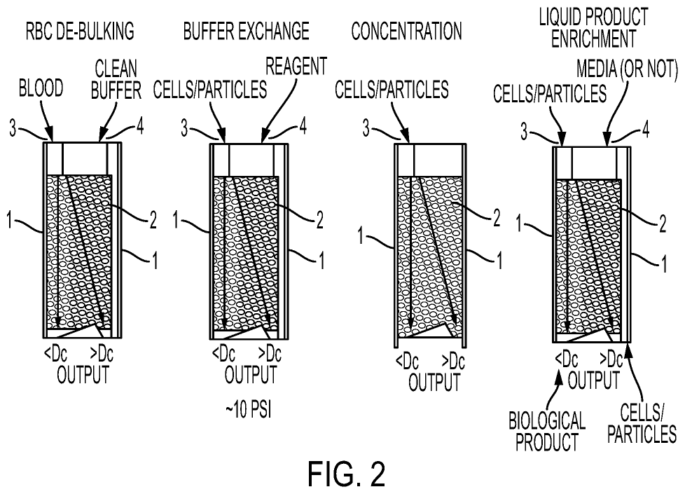

The cartridges described herein may be arranged in a variety of orientations to accomplish different DLD modes or product outcomes (FIG. 2). Four channels are shown in FIG. 2 with side walls (1) and an array of obstacles (2). Samples containing blood, cells or particles enter the channel through a sample inlet at the top (3) and buffer, reagent or media enter the channel at a separate fluid inlet (4). As they flow toward the bottom of the channels, cells or particles with sizes larger than the critical diameter of the array (>Dc) flow at angle that is determined by the array direction of the obstacles and are separated from cells and particles with sizes smaller than the critical diameter of the array (<Dc).

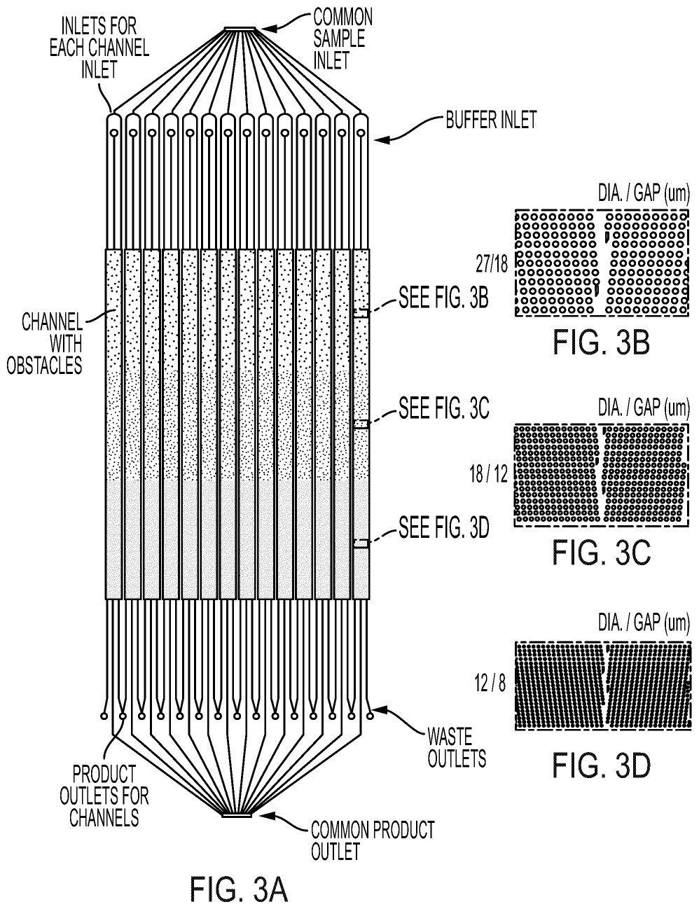

Referring to FIGS. 3A-3D, an embodiment of a cartridge may comprise an arrangement of 14 parallel channels that could be used in a microfluidic device or cartridge. FIGS. 3B-3D illustrate expanded views of sections of the cartridge. In this illustration, the channels have three zone (sections) with progressively smaller gaps. The cartridge has a common sample inlet, e.g., for blood, which feeds the sample to inlets on each channel. There are separate inlets into channels for buffer, but which could, depending processing objectives, be used to introduce fluids with reagents, growth medium or other fluids into channels. At the bottom of each channel there is a product outlet which would typically be used for recovering target cells or particles that have sizes larger than the critical diameter of the obstacle arrays in the channels. The outlets from the individual channels feed into a common product outlet from which the target cells or particles can be recovered. Also shown are waste outlets in which cells and particles with sizes below the critical diameter of the obstacle arrays in the channels exit.

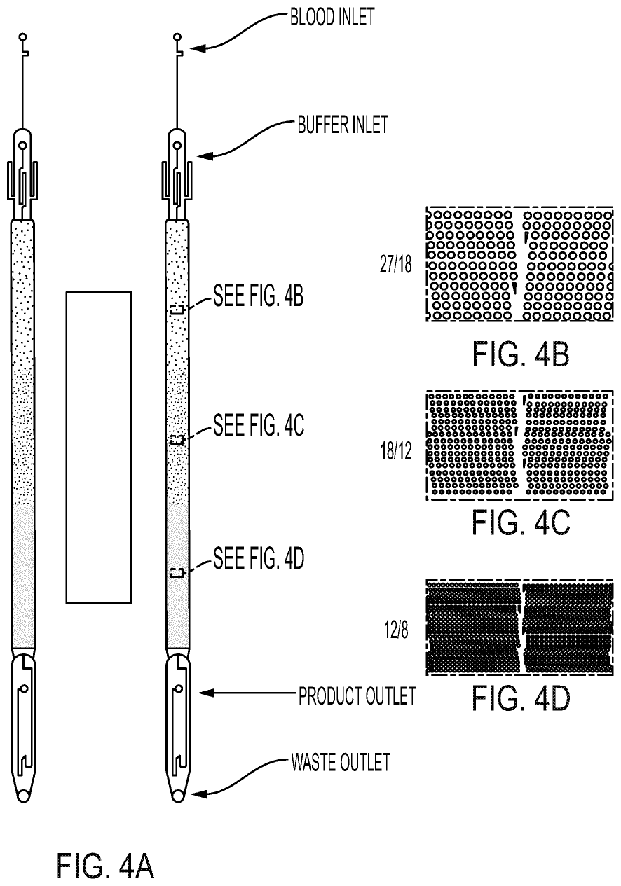

Referring to FIGS. 4A-4D, an embodiment of a cartridge may comprise 2 channels. FIGS. 4B-4D illustrate expanded views of sections of the channels. The channels have three sections designed to have progressively smaller diameter obstacles and gaps.

Some cartridges may have a “bump array” having equilateral triangularly shaped obstacles disposed in a microfluidic channel, as shown in the cross-section diagram of FIG. 5. In the figure, fluid flows in the left-to-right direction, as indicated by the arrow marked, “Fluid.” In this array, equilateral triangular posts are disposed in a parallelogram lattice arrangement that is tilted with respect to the directions of fluid flow. Other lattice arrangements (e.g., square, rectangular, trapezoidal, hexagonal, etc. lattices) can also be used. The tilt angle ε (epsilon) is chosen so the device is periodic. In this embodiment, a tilt angle of 18.4 degrees (1/3 radian) makes the device periodic after three rows. The tilt angle E also represents the angle by which the array direction is offset from the fluid flow direction. The gap between posts is denoted G with equilateral triangle side length S. Streamlines are shown extending between the posts, dividing the fluid flow between the posts into three regions (“stream tubes”) of equal volumetric flow. A relatively large particle (having a size greater than the critical size for the array) follows the array tilt angle when fluid flow is in the direction shown. A relatively small particle (having a size smaller than the critical size for the array) follows the direction of fluid flow.

The cartridges provided herein may comprise arrays of diamond shaped posts as illustrated in FIGS. 6A-6B. FIG. 6A shows a symmetric array of obstacles in which gaps perpendicular to the direction of fluid flow, e.g., Gap 1 (G1), and gaps parallel to the direction of fluid flow, e.g., Gap 2 (G2) are all about the same length. Diamond shaped obstacles may have two diameters, one perpendicular to the direction of fluid flow (P1) and the other parallel to the direction of fluid flow (P2). The right side of the figure shows an asymmetric array in which parallel gaps are shorter than perpendicular gaps. Although, G1 in the asymmetric array has been widened compared to the symmetric array, the reduction in gap G2 results in a critical diameter for the array that is the same as for the symmetrical array. As a result, the two arrays should be about equally effective at separating particles or cells of a given diameter in a sample. However, the widening of G1 allows for a higher sample throughput and reduces channel clogging. FIG. 6B shows, on the left side, an array of diamond obstacles that have been elongated so that their vertical diameter is longer than their horizontal diameter. The middle section of FIG. 6 shows diamond posts that have been elongated so that their horizontal diameter is longer than their vertical diameter and the far-right section of the figure shows hexagonally shaped obstacles that have been horizontally elongated.

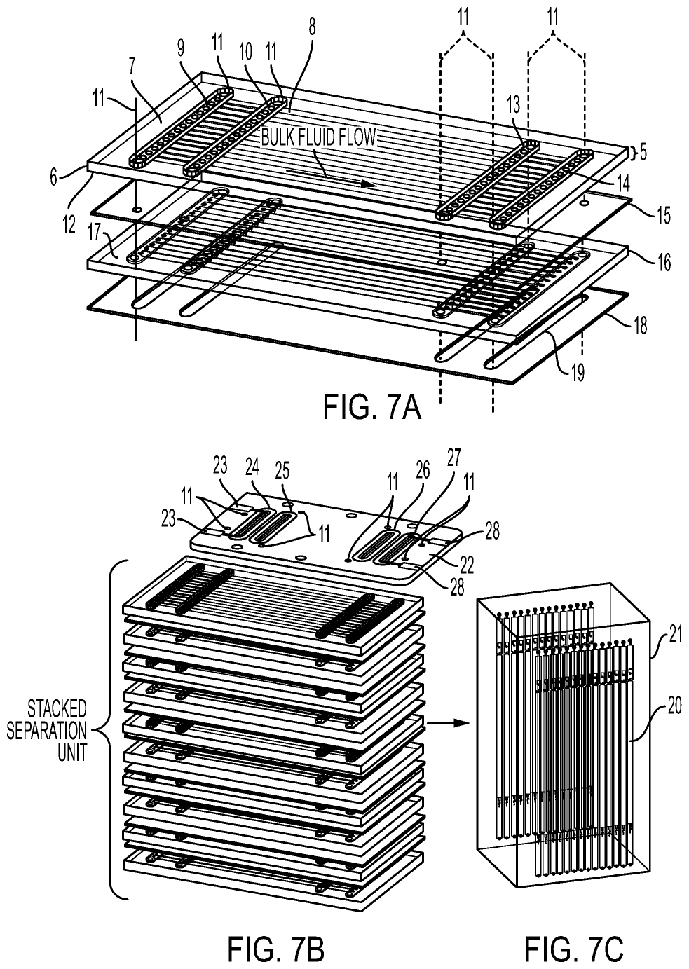

Referring to FIGS. 7A-7C, cartridges describe herein may comprise a stacked separation assembly in which two microfluidic devices or cartridges are combined into a single unit. The topmost device (5) comprises a planar support (6) that may be made using a variety of materials but which is most preferably polymeric and which has a top surface (7) and a bottom surface (12). The top surface of the support (7) contains reservoirs that provide sample inlets (9) and inlets for buffer or other fluid (10) at one end of the support and product outlets (14) and waste outlets (13) at the other end. Each reservoir is fluidically connected through the support using small vias (interior of (9), (10), (13), (14)) that connect the top surface (7) to the channels on the bottom surface (12). The bottom surface of the support (12) has numerous embedded microfluidic channels (8) each of which has an array of obstacles (see FIGS. 1A-1C, 2, 3B-3D, 4B-4D, 5, 6A and 6B and 8B) connected by the channels. The embedded microfluidic layers are bonded to an obstacle bonding layer (15) that seals the first device and prevents fluid from flowing over the obstacles during operation. A second microfluidic device in the stack is shown (16) which contains embedded microfluidic channels on the topmost surface, and is sealed by the same obstacle bonding layer (15) as the topmost device. A reservoir bonding layer (18) is also shown having oblong openings (19) allowing for the passage of liquid to channel inlets and the passage of liquid from channel outlets. The reservoir bonding layer is similar to the obstacle bonding layer except that it attaches to a surface of a device and not obstacles and may be connected to one or more reservoirs feeding the stack of devices or to a manifold. Holes (11) are shown that are used for aligning the stacked devices. As described above, the two embedded microfluidic surfaces face the same obstacle bonding layer. An alternate configuration would be to have the embedded channels on the top surface of both devices, with an intermediate layer between the devices that functions as both an obstacle bonding layer to the embedded channels below and a distribution layer to the reservoirs above. FIG. 7B shows a stack of multiple microfluidic devices that together form a single assembly unit. At the top of this stack (and optionally both at the top and bottom) is a manifold (22) with feeds (23) for the manifold inlet distributor (24) and conduits (28) leading from the manifold product outlet (27). Feeds leading to fluid inlets (25) and conduits for removing fluid from waste outlets (26) would also be present but are not shown in the figure. FIG. 7C shows a stacked separation assembly (20) that has been mounted in a casing (21).

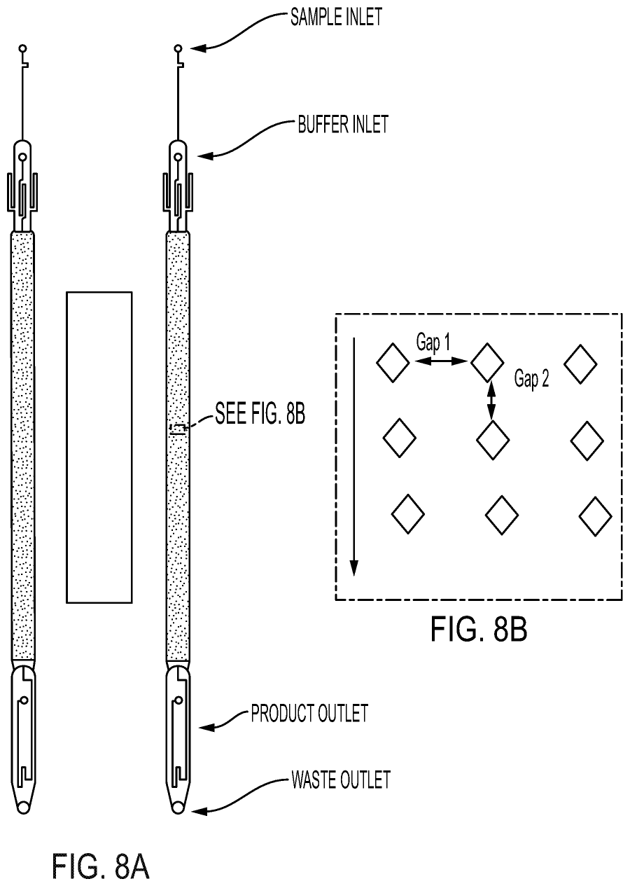

Two channels that might be found in a device depicted in FIG. 7 are shown in FIGS. 8A-8B. An expanded view of a section of the channels is shown in FIG. 8B. In this example, the channel has an array of asymmetrically spaced diamond obstacles, in which G1 is larger than G2. The diamonds are offset so each successive row is shifted laterally relative to the previous row.

The present disclosure provides herein stacked assemblies of microfluidic devices (20) inside a casing (21) which together may be referred to as “cassettes” or a “cassette” (FIG. 9). A port (29) serves as a feed for sample being fed through the casing and to a manifold (22). The port (29) is connected to manifold feeds (23) which distribute sample through manifold sample inlet (24) to channel sample inlets. Once applied, sample flows through channels containing obstacle arrays (see FIGS. 3-6) and product having particles or cells larger than the critical size exit the stack of devices at a manifold product outlet (27). The product then flows from the manifold outlet through product conduits (28) and is conveyed out of the cassette through product outlet port (31). Fluid flows into the cassette and to the manifold through port (51), which is connected to manifold fluid feeds (49). It is distributed by a manifold fluid inlet (25) to channel fluid inlets. The fluid flows through the channel and particles or cells smaller than the critical size exit the stack of devices predominantly through manifold waste outlet (26). These particles or cells then flow through waste conduits (50) that convey waste out of the cassette through outlet port (30).

An embodiment of the cartridges or devices provided herein may comprise a channel bounded by two walls (32), with a sample inlet (33) and a fluid inlet (34) (FIGS. 10A-B). There is a separator wall (35) that prevents the sample flow stream from mixing with the fluid flow stream. The separator wall extends into the obstacle array (36) and ends about halfway down. The arrows in the array show the direction of travel by a target cell with a size larger than the critical size of the array. Initially after entering the obstacle array, the target cells are diverted away from the direction of fluid flow until they reach the separator wall. They then travel along the wall until it ends. Thereafter, they resume being diverted until they exit the channel at the product outlet (37). Particles with sizes smaller than the critical size of the obstacle array are not diverted and exit the channel at the waste outlet (38). FIG. 10B also shows a channel bounded by walls (43) with an inlet for sample (39), an inlet for a reagent (40) and an inlet for buffer or other fluid (42). Sample enters at the inlet and flows onto the obstacle array (44). There, particles or cells larger than the critical diameter of the array are diverted into the reagent stream where they undergo a reaction. A separator wall (41) runs from the reagent inlet part way down the array of obstacles (44) and separates the reagent stream from the stream of buffer or other fluid. This wall maintains the cells or particles in the reagent stream for a longer period of time, thereby providing more time for reaction. At the end of the separator wall, the particles or cells resume being diverted to a product outlet (48) where they may be collected. During this process the cells or particles are separated from unreacted reagent. A second separator wall (45) runs from the end of the first separator wall (41) to a waste outlet (47) where buffer or other fluid, reagent and small particles or cells exit the device and may be collected or discarded. A second waste outlet (46) is used to remove reagent, fluid in which particles or cells in the sample were suspended and particles or cells smaller than the critical diameter of the obstacle array. These materials may be recovered or discarded.

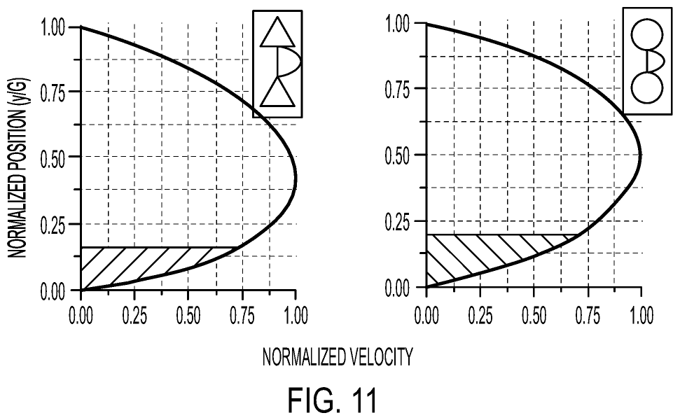

A comparison of normalized velocity flow between two equilateral triangular posts (left panel) and normalized velocity flow between two circular posts (right panel) can be made (FIG. 11), demonstrating the effect of obstacle or post shape. The shaded portions of FIG. 11 represent an equal proportion of area-under-the-curve, demonstrating that the critical radius for particles flowing past the point of the triangle is significantly smaller (<15% gap width) than the critical radius for particles flowing past the round post (>20% gap width).

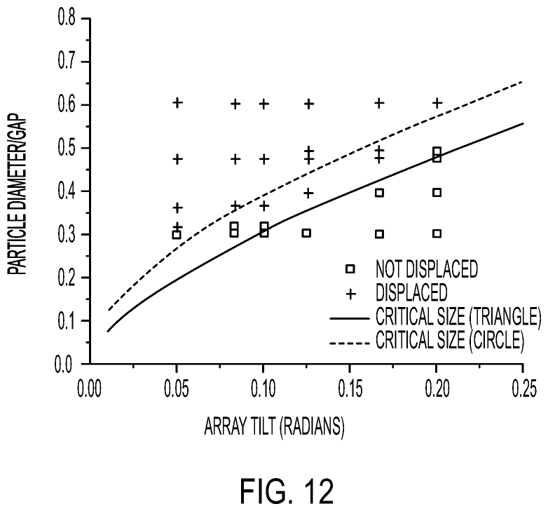

FIG. 12 is a graph of predicted critical diameter versus the array tilt angle (‰) for arrays of triangular (lower line) and circular (upper line) obstacles. The analysis of FIG. 12 further demonstrates the affect of post shape in displacing particles or cells shown in FIG. 11.

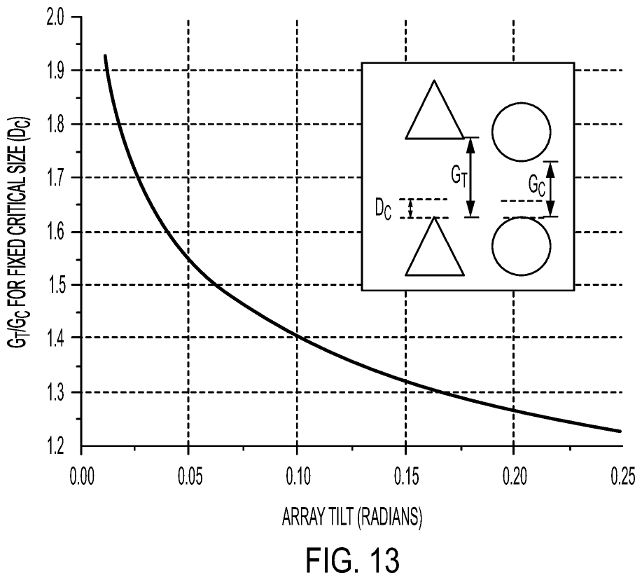

Referring to FIG. 13, the effect of the tilt angle (“Array Tilt” in the figure) on gap length G may be illustrated. G_{T} refers to the gap length between triangular posts, and G_{C} refers to the gap length between round posts. As the array tilt increases, the difference in gap lengths required for a particular critical size of the array (D_{C}), between triangular and circular posts, decreases.

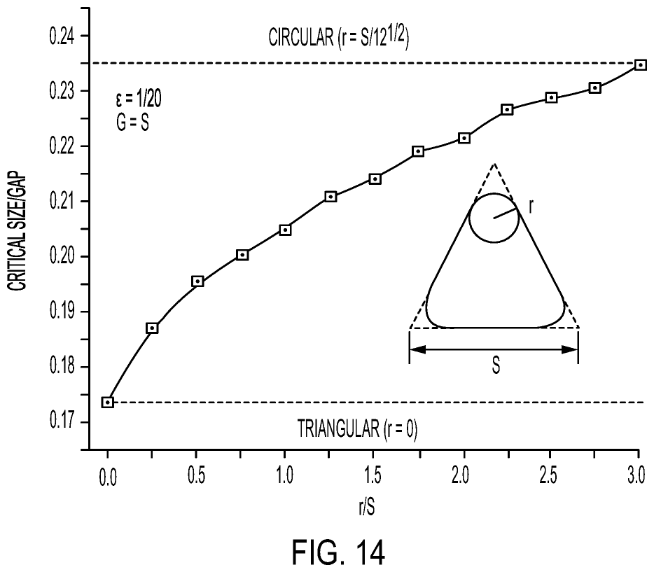

The effect of obstacle edge roundness (expressed as r/S) on the critical size exhibited on the side of a gap bounded by the edge is illustrated in FIG. 14. Increasing rounded of a post increases the critical size value of that post for a given gap length.

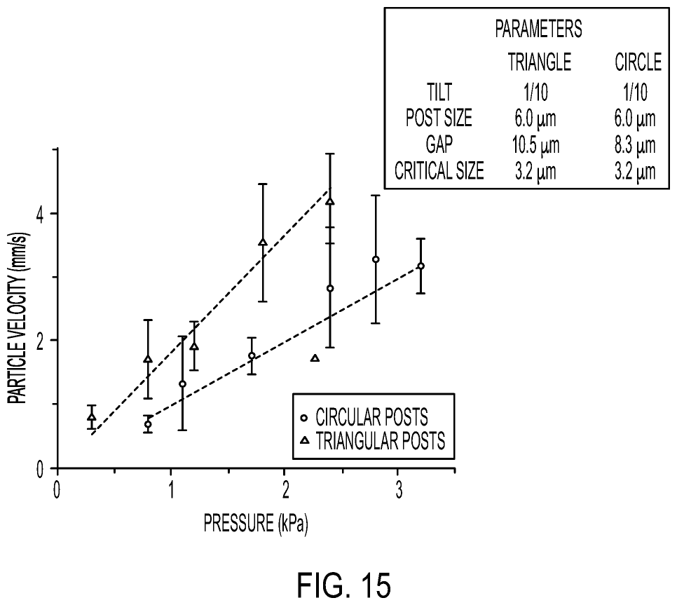

In addition to critical size, posts of different shapes may also affect particle velocity given constant applied pressure. FIG. 15 illustrates the effect of applied pressure on particle velocity in bump arrays having triangular posts (data shown as triangles) and bump arrays having circular posts (data shown as circles). Given an applied pressure, arrays with triangular posts will result in a larger particle velocity than those with circular posts. Furthermore, the rate of particle velocity increase upon increasing pressure is also greater in triangular post arrays than circular post arrays.

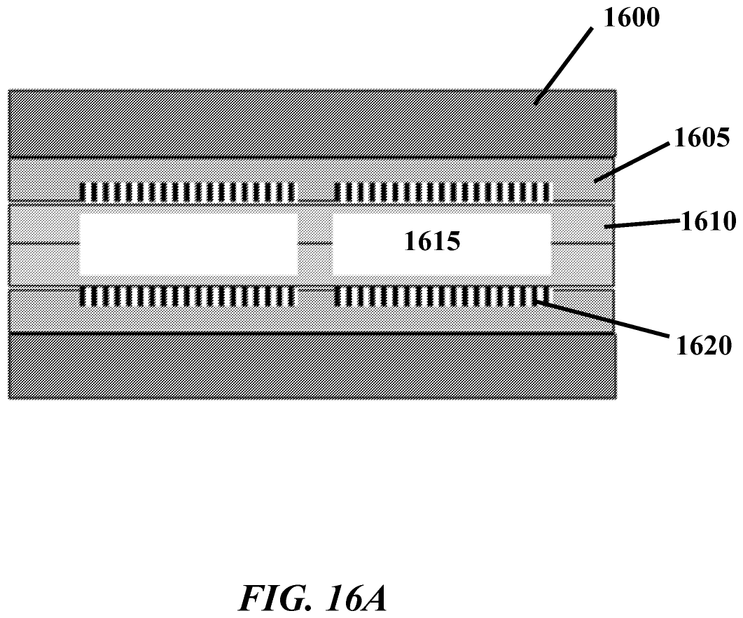

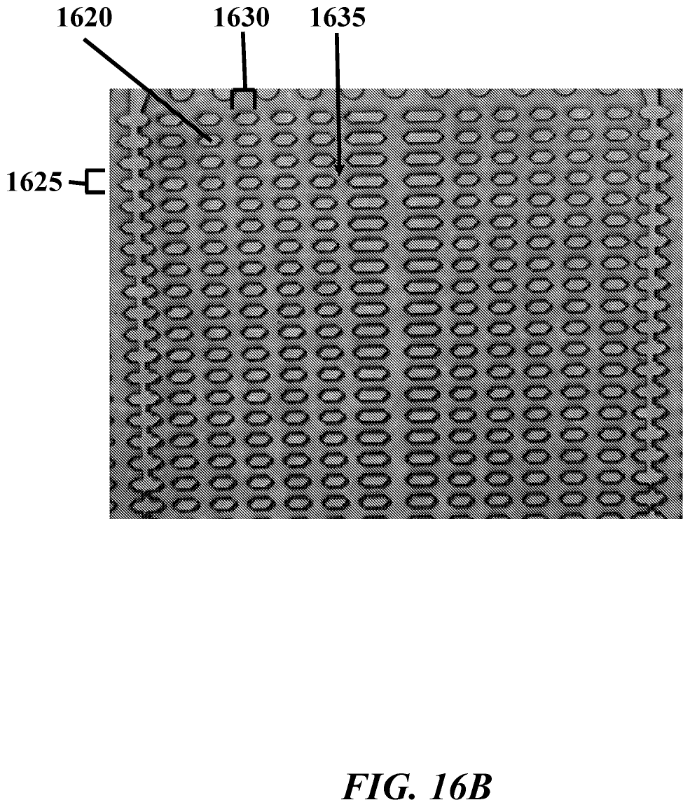

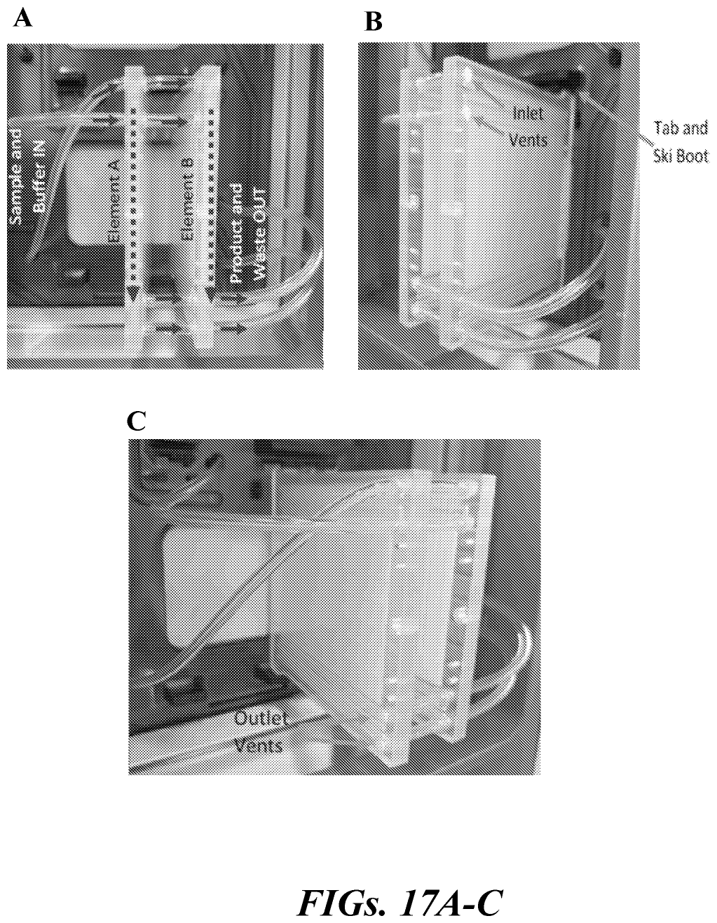

Referring to FIG. 16A, the cartridges described herein comprise a Seal/Lid 1600 on the top and/or bottom and a separation layer 1605, that comprises a plurality of obstacles 1620 that promote separation, a fluidic layer 1610, and a void space or crumple zone that allows fabrication of the cartridge without deforming the plurality of obstacles. Referring to FIG. 16B the plurality of obstacles 1620 may be arrayed in rows 1625 and columns 1630, such that gaps 1635 configured to allow the passage of fluid and cells are formed. The obstacles may be arrayed such that they are stacked with no or minimal offset between repeating rows. Referring to FIGS. 17A to C two or more cartridges may be stacked or connected in series or parallel to achieve greater separation or higher throughput.

As similar devices or microfluidic cartridges operate on a sub-millimeter scale and handles micro-liters, nano-liters, or smaller quantities of fluids, a major obstacle in manufacturing is avoiding damage or deformation of obstacles during embossing or assembly. For example, handling of the chip may result in pressure to the planar support, especially when planar supports are pressed together, which may then result in deformation or destruction of the planar support(s), obstacles (i.e. an array of obstacles), and the various separation lanes. Such deformation or destruction may result in a significant loss of performance in purifying particles or cells or may completely compromise the function of the microfluidic cartridge. In order to avoid potential deformations and defects during manufacturing and assembly, other microfluidic systems require slower manufacturing runs or accept diminished performance.

In an aspect, the present disclosure provides a microfluidic cartridge for purifying cells or particles. The microfluidic cartridge may include a first planar support. The first planar support may comprise a top surface and a bottom surface. The device may include a second planar support. The second planar support may comprise a top surface and a bottom surface. A top surface may comprise at least one embedded channel extending from one or more inlets to one or more outlets. The at least one embedded channel may comprise an array of obstacles. The bottom surface of the first and second planar support may comprise a void space. The void space may be configured to be deformed when the bottom of the first planar support is pressed to the bottom of the second planar support.

Separation according to this description occurs along a channel embedded in a planar support, the channel comprising a plurality of obstacles. For cartridges of this description a first and a second planar surface may be utilized. The first and second planar surfaces may be stacked (e.g., bottom to bottom or top to bottom with a spacer doubling the throughput and separation capacity while maintaining a small footprint. A top surface of a first and/or second planar surface may comprise at least 1 embedded channel to about 500 embedded channels. A top surface may comprise at least 1 embedded channel to about 2 embedded channels, 1 embedded channel to about 5 embedded channels, 1 embedded channel to about 20 embedded channels, 1 embedded channel to about 50 embedded channels, 1 embedded channel to about 100 embedded channels, 1 embedded channel to about 500 embedded channels, about 2 embedded channels to about 5 embedded channels, about 2 embedded channels to about 20 embedded channels, about 2 embedded channels to about 50 embedded channels, about 2 embedded channels to about 100 embedded channels, about 2 embedded channels to about 500 embedded channels, about 5 embedded channels to about 20 embedded channels, about 5 embedded channels to about 50 embedded channels, about 5 embedded channels to about 100 embedded channels, about 5 embedded channels to about 500 embedded channels, about 20 embedded channels to about 50 embedded channels, about 20 embedded channels to about 100 embedded channels, about 20 embedded channels to about 500 embedded channels, about 50 embedded channels to about 100 embedded channels, about 50 embedded channels to about 500 embedded channels, or about 100 embedded channels to about 500 embedded channels. A top surface may comprise at least 1 embedded channel, about 2 embedded channels, about 5 embedded channels, about 20 embedded channels, about 50 embedded channels, about 100 embedded channels, or about 500 embedded channels. A top surface may comprise at least 1 embedded channel, about 2 embedded channels, about 5 embedded channels, about 20 embedded channels, about 50 embedded channels, or about 100 embedded channels. A top surface may comprise at least at most about 2 embedded channels, about 5 embedded channels, about 20 embedded channels, about 50 embedded channels, about 100 embedded channels, or about 500 embedded channels. A top surface or a first or second planar surface may comprise about 28 channels (56 when stacked). An additional third, fourth, fifth, or sixth planar surface may also comprise a similar amount of embedded channels as the first or second planar surface.

The microfluidic cartridge may comprise at least 1 inlet to about 50 inlets. The microfluidic cartridge may comprise at least 1 inlet to about 2 inlets, 1 inlet to about 5 inlets, 1 inlet to about 10 inlets, 1 inlet to about 20 inlets, 1 inlet to about 50 inlets, about 2 inlets to about 5 inlets, about 2 inlets to about 10 inlets, about 2 inlets to about 20 inlets, about 2 inlets to about 50 inlets, about 5 inlets to about 10 inlets, about 5 inlets to about 20 inlets, about 5 inlets to about 50 inlets, about 10 inlets to about 20 inlets, about 10 inlets to about 50 inlets, or about 20 inlets to about 50 inlets. The microfluidic cartridge may comprise at least 1 inlet, about 2 inlets, about 5 inlets, about 10 inlets, about 20 inlets, or about 50 inlets. The microfluidic cartridge may comprise at least 1 inlet, about 2 inlets, about 5 inlets, about 10 inlets, or about 20 inlets. The microfluidic cartridge may comprise at least at most about 2 inlets, about 5 inlets, about 10 inlets, about 20 inlets, or about 50 inlets. The inlets may be fed by a common fluidic system or a dual fluidic system (one for buffer/diluent and one for sample).

The microfluidic cartridge may comprise at least 1 outlet to about 50 outlets. The microfluidic cartridge may comprise at least 1 outlet to about 2 outlets, 1 outlet to about 5 outlets, 1 outlet to about 10 outlets, 1 outlet to about 20 outlets, 1 outlet to about 50 outlets, about 2 outlets to about 5 outlets, about 2 outlets to about 10 outlets, about 2 outlets to about 20 outlets, about 2 outlets to about 50 outlets, about 5 outlets to about 10 outlets, about 5 outlets to about 20 outlets, about 5 outlets to about 50 outlets, about 10 outlets to about 20 outlets, about 10 outlets to about 50 outlets, or about 20 outlets to about 50 outlets. The microfluidic cartridge may comprise at least 1 outlet, about 2 outlets, about 5 outlets, about 10 outlets, about 20 outlets, or about 50 outlets. The microfluidic cartridge may comprise at least 1 outlet, about 2 outlets, about 5 outlets, about 10 outlets, or about 20 outlets. The microfluidic cartridge may comprise at least at most about 2 outlets, about 5 outlets, about 10 outlets, about 20 outlets, or about 50 outlets. The outlets may feed a common fluidic system or a dual fluidic system (one for waste and one for enriched target cells or particles).

The cartridge comprising two or more planar surfaces may comprise a void space to protect the array of obstacles in the lanes as their small size leads their susceptibility to deformation, leading to malfunction.

The void space of the microfluidic cartridge may be configured to deform, bend, swell, collapse, or crumple. The void space may be configured to protect the obstacles, channels, inlets, outlets, planar surfaces, or any combination thereof, from damage, displacement, deformation, or malfunction. The void space may comprise a crumple zone that is configured to protect the obstacles, channels, inlets, outlets, planar surfaces, or any combination thereof, from damage, displacement, deformation, or malfunction. The void space may have a volume of about 1 cubic μm to about 10,000 cubic μm. The void space may have a volume of about 1 cubic μm to about 5 cubic μm, about 1 cubic μm to about 10 cubic μm, about 1 cubic μm to about 30 cubic μm, about 1 cubic μm to about 50 cubic μm, about 1 cubic μm to about 100 cubic μm, about 1 cubic μm to about 300 cubic μm, about 1 cubic μm to about 1,000 cubic μm, about 1 cubic μm to about 3,000 cubic μm, about 1 cubic μm to about 10,000 cubic μm, about 5 cubic μm to about 10 cubic μm, about 5 cubic μm to about 30 cubic μm, about 5 cubic μm to about 50 cubic μm, about 5 cubic μm to about 100 cubic μm, about 5 cubic μm to about 300 cubic μm, about 5 cubic μm to about 1,000 cubic μm, about 5 cubic μm to about 3,000 cubic μm, about 5 cubic μm to about 10,000 cubic μm, about 10 cubic μm to about 30 cubic μm, about 10 cubic μm to about 50 cubic μm, about 10 cubic μm to about 100 cubic μm, about 10 cubic μm to about 300 cubic μm, about 10 cubic μm to about 1,000 cubic μm, about 10 cubic μm to about 3,000 cubic μm, about 10 cubic μm to about 10,000 cubic μm, about 30 cubic μm to about 50 cubic μm, about 30 cubic μm to about 100 cubic μm, about 30 cubic μm to about 300 cubic μm, about 30 cubic μm to about 1,000 cubic μm, about 30 cubic μm to about 3,000 cubic μm, about 30 cubic μm to about 10,000 cubic μm, about 50 cubic μm to about 100 cubic μm, about 50 cubic μm to about 300 cubic μm, about 50 cubic μm to about 1,000 cubic μm, about 50 cubic μm to about 3,000 cubic μm, about 50 cubic μm to about 10,000 cubic μm, about 100 cubic μm to about 300 cubic μm, about 100 cubic μm to about 1,000 cubic μm, about 100 cubic μm to about 3,000 cubic μm, about 100 cubic μm to about 10,000 cubic μm, about 300 cubic μm to about 1,000 cubic μm, about 300 cubic μm to about 3,000 cubic μm, about 300 cubic μm to about 10,000 cubic μm, about 1,000 cubic μm to about 3,000 cubic μm, about 1,000 cubic μm to about 10,000 cubic μm, or about 3,000 cubic μm to about 10,000 cubic μm. The void space may have a volume of about 1 cubic μm, about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm. The void space may have a volume of at least about 1 cubic μm, about 5 cubic μm, about 10 cubic μm, about 30 cubic μm, about 50 cubic μm, about 100 cubic μm, about 300 cubic μm, about 1,000 cubic μm, or about 3,000 cubic μm. The void space may have a volume of at most about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm. The void space maybe about X cubic μm.

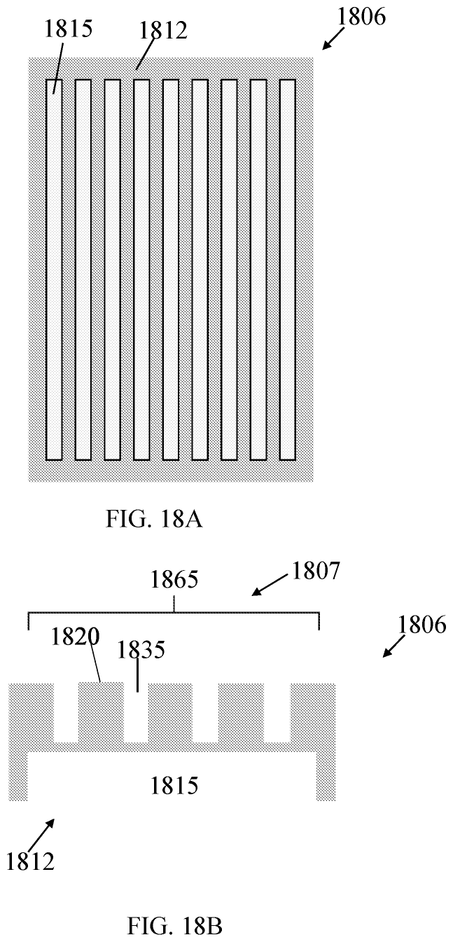

Referring to FIG. 18A, which shows a non-limiting view of a bottom surface 1812 of a planar support 1806 of this disclosure. The bottom surface may comprise a plurality of void spaces 1815, shown here arranged into strips that run parallel with the length of the planar support. The void spaces run beneath the array or column of obstacles (not shown) or the lanes formed by the columns of obstacles (not shown) fabricated on the top surface of the planar support. Referring to FIG. 18B a cross-sectional view of a planar support 1806 is shown. The top surface of the planar support 1807 comprises a plurality of individual obstacles 1820 formed into arrays or columns creating gaps 1835 to allow the flow of fluid, cells, and/or particles. Beneath the obstacles embedded in the bottom surface of the planar support 1812 is a void space 1815. The area of the void space (length×width) opposite the lane can be at least about 80% of the area (length×width) of the lane. In certain embodiments, the area of the void space (length×width) opposite the lane can be at least about 90%, 100%, 110%, or 120% up to and including about 150% of the area (length×width) of the lane.

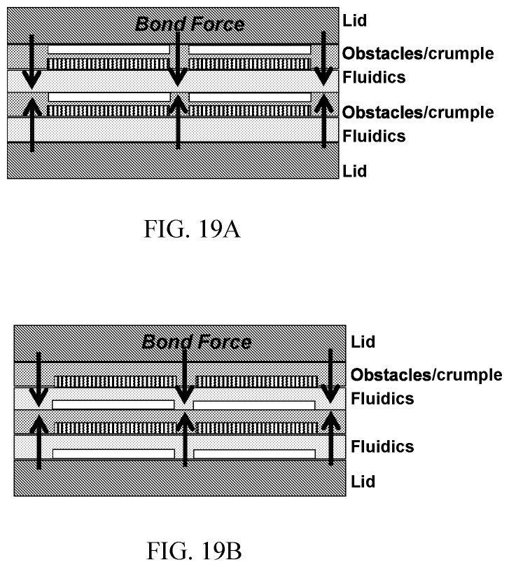

In one configuration the void spaces of the two planar supports are symmetrical or nearly symmetrical. And pressed back to back as shown in FIG. 16A. However as shown in FIG. 19 alternative arrangements are shown. Such cases the supports are not pressed back to back but stacked and the void space is above, as in 19A or, below, as in 19B, the obstacle layer.

The void space may be separated into two or more void spaces. The void space may be separated into at least 3, 4, 5, 6, 7, 8, 9, 10, 11, 12, 13, 14, 15, 16, 17, 18, 19, 20, 21, 22, 23, 24, 25, 26, 27, 28, 29, or 30 void spaces. The void space may be separated into exactly two void spaces. There may be a 1:1 ratio between channels or lanes and void spaces for each planar support comprising obstacles.

The planar support may be fabricated from two layers of material bonded together. The layers may be bonded together by adhesive, polymer, or thermoplastic. The layers may be comprised of polymer or thermoplastic. The polymer or thermoplastic layers or bonding material may be comprised of high-density polyethylene (HDPE), polypropylene (PP), polyethylene terephthalate (PT), polycarbonate (PC), or cyclic olefin copolymer (COC).

The top layer of a cartridge may comprise an array of obstacles in at least one embedded channel, void space, at least one inlet, at least one outlet, or combination thereof. The bottom layer of a cartridge may comprise an array of obstacles in at least one embedded channel, void space, at least one inlet, at least one outlet, or combination thereof. The layers may be positioned to where the planar supports are bonded together on their side surfaces, bottom surfaces, or top surfaces. The void space may be inside the interface of the planar supports bonded together, or outside the interface.

The microfluidic cartridge may further comprise an obstacle bonding layer that is bonded to the surface of the planar support and a top surface of the array of obstacles in the embedded channels to prevent fluid or sample from flowing over the array of obstacles during operation of the cartridge. The obstacle bonding layer may be metallic, polymer, or thermoplastic. The obstacle bonding layer may be a cover or a film. The polymer or thermoplastic layers or bonding material may be comprised of high-density polyethylene (HDPE), polypropylene (PP), polyethylene terephthalate (PT), polycarbonate (PC), or cyclic olefin copolymer (COC). The microfluidic cartridge may comprise two obstacle bonding layers on the outside of the top planar support. The microfluidic cartridge may comprise a single obstacle bonding layer in the middle of the cartridge as the bonding agent for the planar supports. The obstacle bonding layer may comprise one or more passages fluidically connected to the one or more inlets of the embedded channels which permit the flow of sample into the channels and one or more passages fluidically connected to the one or more outlets of the channels that permit the flow of fluid out from the one or more outlets. Such an obstacle layer may comprise at least about 1, at least about 2, at least about 3, at least about 4, at least about 5, at least about 10, at least about 20, at least about 30, at least about 50, or at least about 100 passages fluidically connected to the one or more inlets or one or more outlets of the embedded channels.

The microfluidic cartridge may have the obstacles positioned so as to define a critical size of the cartridge such that when a sample is applied to an inlet of the cartridge and flows to an outlet, particles or cells in the sample larger than the critical size are separated from particles or cells in the sample smaller than the critical size. Each obstacle may have its own individual sub-critical size, the sum the individual obstacles defining the critical size of the cartridge. The one or more outlets of the cartridge may comprise at least one product outlet, wherein target particles or cells, having a size larger than the critical size of the cartridge, are directed to the at least one product outlet. The one or more outlets of the cartridge may comprise at least one product outlet, wherein target particles or cells, having a size smaller than the critical size of the cartridge, are directed to the at least one product outlet. The cartridge may have at least about 1, at least about 2, at least about 3, at least about 5, at least about 10, or at least about 50 product outlets. The one or more outlets may comprise at least one waste outlet. The contaminants, particles, or cells, having a size smaller than the critical size, may flow to the at least one waste outlet. The contaminants, particles, or cells, having a size larger than the critical size, may flow to the at least one waste outlet. The cartridge may have at least about 1, at least about 2, at least about 3, at least about 5, at least about 10, or at least about 50 waste outlets.

The obstacles used in the cartridge may take the shape of columns or be triangular, square, rectangular, diamond shaped, trapezoidal, hexagonal, teardrop shaped, circular shape, semicircular shape, triangular with top side horizontal shape, and triangular with bottom side horizontal shape. In addition, adjacent obstacles may have a geometry such that the portions of the obstacles defining the gap are either symmetrical or asymmetrical about the axis of the gap that extends in the direction of bulk fluid flow. The obstacles may have vertices that extend into parallel gaps such that the gaps are flanked on either side by one or more vertices pointing toward one another but not directly opposite one another. The obstacles may have vertices that extend into perpendicular gaps such that the gaps are flanked on either side by vertices pointing toward one another and that are directly opposite one another. Obstacle location and shape can vary in a single chip. Additional obstacles can be added to any location of the device for any specific requirement. Also, the shape of the obstacle can be different in a device. Any combinations of posts shape, size and location can be used for specific requirement. The cartridge may be comprised of only diamond or hexagonal shaped obstacles.

The obstacle shapes may be elongated perpendicularly to the direction of fluid flow such that they have a horizontal length (P1) that is different from their vertical length (P2). P1 may have a length of about 1 μm to about 160 μm. P1 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P1 may have a length of about 1 μm, about 10 μm, about 15 μm, about 30 μm, about 40 μm, about 80 μm, or about 160 μm. P1 may have a length of at least about 1 μm, about 10 μm, about 15 μm, about 30 μm, about 40 μm, or about 80 μm. P1 may have a length of at most about 10 μm, about 15 μm, about 30 μm, about 40 μm, about 80 μm, or about 160 μm. P2 may have a length of about 1 μm to about 160 μm. P2 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P2 may have a length of about 1 μm, about 10 μm, about 15 μm, about 30 μm, about 40 μm, about 80 μm, or about 160 μm. P2 may have a length of at least about 1 μm, about 10 μm, about 15 μm, about 30 μm, about 40 μm, or about 80 μm. P2 may have a length of at most about 10 μm, about 15 μm, about 30 μm, about 40 μm, about 80 μm, or about 160 μm. P1 may be longer than P2 by about 25% to about 200%. P1 may be longer than P2 by about 25% to about 50%, about 25% to about 75%, about 25% to about 100%, about 25% to about 150%, about 25% to about 200%, about 50% to about 75%, about 50% to about 100%, about 50% to about 150%, about 50% to about 200%, about 75% to about 100%, about 75% to about 150%, about 75% to about 200%, about 100% to about 150%, about 100% to about 200%, or about 150% to about 200%. P1 may be longer than P2 by about 25%, about 50%, about 75%, about 100%, about 150%, or about 200%. P1 may be longer than P2 by at least about 25%, about 50%, about 75%, about 100%, or about 150%. P1 may be longer than P2 by at most about 50%, about 75%, about 100%, about 150%, or about 200%.

The microfluidic cartridge may comprise obstacles as an array of obstacles. The obstacles may be arranged in in columns and in rows that form discreet arrays. The array of obstacles may compromise at least about 5 columns to about 50 columns. The array of obstacles may compromise at least about 5 columns to about 10 columns, about 5 columns to about 28 columns, about 5 columns to about 29 columns, about 5 columns to about 30 columns, about 5 columns to about 50 columns, about 10 columns to about 28 columns, about 10 columns to about 29 columns, about 10 columns to about 30 columns, about 10 columns to about 50 columns, about 28 columns to about 29 columns, about 28 columns to about 30 columns, about 28 columns to about 50 columns, about 29 columns to about 30 columns, about 29 columns to about 50 columns, or about 30 columns to about 50 columns. The array of obstacles may compromise at least about 5 columns, about 10 columns, about 28 columns, about 29 columns, about 30 columns, or about 50 columns. The array of obstacles may compromise at least about 5 columns, about 10 columns, about 28 columns, about 29 columns, or about 30 columns. The array of obstacles may compromise at least at most about 10 columns, about 28 columns, about 29 columns, about 30 columns, or about 50 columns. The array of obstacles may compromise at least about 20 rows to about 500 rows. The array of obstacles may compromise at least about 20 rows to about 30 rows, about 20 rows to about 60 rows, about 20 rows to about 100 rows, about 20 rows to about 200 rows, about 20 rows to about 500 rows, about 30 rows to about 60 rows, about 30 rows to about 100 rows, about 30 rows to about 200 rows, about 30 rows to about 500 rows, about 60 rows to about 100 rows, about 60 rows to about 200 rows, about 60 rows to about 500 rows, about 100 rows to about 200 rows, about 100 rows to about 500 rows, or about 200 rows to about 500 rows. The array of obstacles may compromise at least about 20 rows, about 30 rows, about 60 rows, about 100 rows, about 200 rows, or about 500 rows. The array of obstacles may compromise at least about 20 rows, about 30 rows, about 60 rows, about 100 rows, or about 200 rows. The array of obstacles may compromise at least at most about 30 rows, about 60 rows, about 100 rows, about 200 rows, or about 500 rows. Multiple arrays of obstacles can be arranged in discrete lanes. The array of obstacles of the first or second planar support forms about 10 lanes to about 50 lanes. The array of obstacles of the first or second planar support forms about 10 lanes to about 20 lanes, about 10 lanes to about 28 lanes, about 10 lanes to about 30 lanes, about 10 lanes to about 50 lanes, about 20 lanes to about 28 lanes, about 20 lanes to about 30 lanes, about 20 lanes to about 50 lanes, about 28 lanes to about 30 lanes, about 28 lanes to about 50 lanes, or about 30 lanes to about 50 lanes. The array of obstacles of the first or second planar support forms about 10 lanes, about 20 lanes, about 28 lanes, about 30 lanes, or about 50 lanes. The array of obstacles of the first or second planar support forms at least about 10 lanes, about 20 lanes, about 28 lanes, or about 30 lanes. The array of obstacles of the first or second planar support forms at most about 20 lanes, about 28 lanes, about 30 lanes, or about 50 lanes.

Each cartridge may comprise at least one, at least two, at least three, or at least four sets of arrays of obstacles. Each planar top surface may comprise at least one or at least two arrays. The cartridge may comprise a total of about 20 lanes to about 100 lanes. The cartridge may comprise a total of about 20 lanes to about 40 lanes, about 20 lanes to about 56 lanes, about 20 lanes to about 60 lanes, about 20 lanes to about 100 lanes, about 40 lanes to about 56 lanes, about 40 lanes to about 60 lanes, about 40 lanes to about 100 lanes, about 56 lanes to about 60 lanes, about 56 lanes to about 100 lanes, or about 60 lanes to about 100 lanes. The cartridge may comprise a total of about 20 lanes, about 40 lanes, about 56 lanes, about 60 lanes, or about 100 lanes. The cartridge may comprise a total of at least about 20 lanes, about 40 lanes, about 56 lanes, or about 60 lanes. The cartridge may comprise a total of at most about 40 lanes, about 56 lanes, about 60 lanes, or about 100 lanes.

The inlets, outlets, or both, of the microfluidic cartridge may be in fluid connection with pumps or motors to drive the flow of fluids within and outside of the cartridge. The inlets, outlets, or both, may be fluidically connected to at least about 1, 2, 3, 4, 5, 6, 7, 8, 9, or 10 pumps. The pumps may be peristaltic pumps. The pumps may be fluidically connected to each other or isolated. The inlets and outlets of the cartridge may be in fluidic connection with two peristaltic pumps connected in parallel to each other. The inlets and outlets of the cartridge may be in fluidic connection with two peristaltic pumps connected in serial to each other.

The microfluidic cartridge may be fabricated from a metal, polymer, or thermoplastic. The polymer or thermoplastic may be comprised of high-density polyethylene (HDPE), polypropylene (PP), polyethylene terephthalate (PT), polycarbonate (PC), or cyclic olefin copolymer (COC). In an example, the microfluidic cartridge is comprised of cyclic olefin copolymer.

The present disclosure also provides for a microfluid assembly comprising a plurality of microfluidic cartridges in fluidic connection. The cartridges in the assembly may be stacked or layered. The plurality of microfluidic cartridges may comprise at least about 2, 3, 4, 5, 6, 7, 8, 9, 10, 20, or 30 cartridges. The plurality of cartridges may be fluidically connected in serial or in parallel.

Cells, e.g., cells in compositions prepared by apheresis or leukapheresis, may be isolated by performing DLD using microfluidic cartridges that have a channel through which fluid flows from inlets at one end to outlets at the opposite end. Basic principles of size based microfluidic separations and the design of obstacle arrays for separating cells have been provided elsewhere (see, US 2014/0342375; US 2016/0139012; U.S. Pat. Nos. 7,318,902 and 7,150,812, which are hereby incorporated herein in their entirety) and are also summarized in the sections below.

During DLD, a fluid sample containing cells is introduced into a device at an inlet and is carried along with fluid flowing through the device to outlets. As cells in the sample traverse the device, they encounter posts or other obstacles that have been positioned to form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for leukapheresis-derived compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds to that cell in a way that promotes DLD separation and which thereby results in a complex that is larger than uncomplexed leukocytes. It may then be possible to carry out a separation on a device having a critical size smaller than the complexes but bigger than the uncomplexed cells.

### II. Making and Operating Microfluidic Devices

General procedures for making and using microfluidic devices that are capable of separating cells on the basis of size are well known in the art. Such devices include those described in U.S. Pat. Nos. 5,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 each of which is also incorporated by reference herein in its entirety. Of the various references describing the making and use of devices, U.S. Pat. No. 7,150,812 provides particularly good guidance and 7,735,652 is of particular interest with respect to microfluidic devices for separations performed on samples with cells found in blood (in this regard, see also US 2007/0160503).

A device can be made using any of the materials from which micro- and nano-scale fluid handling devices are typically fabricated, including silicon, glasses, plastics, and hybrid materials. A diverse range of thermoplastic materials suitable for microfluidic fabrication is available, offering a wide selection of mechanical and chemical properties that can be leveraged and further tailored for specific applications. In an aspect, the microfluidic cartridge may be fabricated by soft embossing and UV-light curing.

The microfluidic cartridge (or device, cassette, chip, etc.) may be made by techniques including Replica molding, Soft lithography with PDMS, Thermoset polyester, Embossing, soft embossing, hot embossing, Roll to Roll embossing, Injection Molding, Laser Ablation, UV-light curing, and combinations thereof. Further details can be found in “Disposable microfluidic devices: fabrication, function and application” by Fiorini, et al. (BioTechniques 38:429-446 (March 2005)), which is hereby incorporated by reference herein in its entirety. The book “Lab on a Chip Technology” edited by Keith E. Herold and Avraham Rasooly, Caister Academic Press Norfolk UK (2009) is another resource for methods of fabrication and is hereby incorporated by reference herein in its entirety.

High-throughput embossing methods such as reel-to-reel processing of thermoplastics is an attractive method for industrial microfluidic chip production. The use of single chip hot embossing can be a cost-effective technique for realizing high-quality microfluidic devices during the prototyping stage. Methods for the replication of microscale features in two thermoplastics, polymethylmethacrylate (PMMA) and/or polycarbonate (PC), are described in “Microfluidic device fabrication by thermoplastic hot-embossing” by Yang, et al. (Methods Mol. Biol. 949: 115-23 (2013)), which is hereby incorporated by reference herein in its entirety

The flow channel can be constructed using two or more pieces which, when assembled, form a closed cavity (preferably one having orifices for adding or withdrawing fluids) having the obstacles disposed within it. The obstacles can be fabricated on one or more pieces that are assembled to form the flow channel, or they can be fabricated in the form of an insert that is sandwiched between two or more pieces that define the boundaries of the flow channel.

The obstacles may be solid bodies that extend in an array laterally across the flow channel and longitudinally along the channel from the inlets to the outlets. Where an obstacle is integral with (or an extension of) one of the faces of the flow channel at one end of the obstacle, the other end of the obstacle can be sealed to or pressed against the opposite face of the flow channel. A small space (preferably too small to accommodate any particles of interest for an intended use) is tolerable between one end of an obstacle and a face of the flow channel, provided the space does not adversely affect the structural stability of the obstacle or the relevant flow properties of the device.

Surfaces can be coated to modify their properties and polymeric materials employed to fabricate devices, can be modified in many ways. In some cases, functional groups such as amines or carboxylic acids that are either in the native polymer or added by means of wet chemistry or plasma treatment are used to crosslink proteins or other molecules. DNA can be attached to COC and PMMA substrates using surface amine groups. Surfactants such as Pluronic® can be used to make surfaces hydrophilic and protein repellant by adding Pluronic® to PDMS formulations. In some cases, a layer of PMMA is spin coated on a device, e.g., microfluidic chip and PMMA is “doped” with hydroxypropyl cellulose to vary its contact angle.

To reduce non-specific adsorption of cells or compounds, e.g., released by lysed cells or found in biological samples, onto the channel walls, one or more walls may be chemically modified to be non-adherent or repulsive. The walls may be coated with a thin film coating (e.g., a monolayer) of commercial non-stick reagents, such as those used to form hydrogels. Additional examples of chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls can depend on the nature of the species being repelled and the nature of the walls and the species being attached. Such surface modification techniques are well known in the art. The walls may be functionalized before or after the device is assembled.

### III. CAR T and NK Cells

Methods for making and using CAR T and natural killer (NK) cells are well known in the art. Procedures have been described in, for example, U.S. Pat. Nos. 9,629,877; 9,328,156; 8,906,682; US 2017/0224789; US 2017/0166866; US 2017/0137515; US 2016/0361360; US 2016/0081314; US 2015/0299317; and US 2015/0024482; each of which is incorporated by reference herein in its entirety.

The present disclosure provides microfluidic cartridges (i.e. devices, chips, cassettes, plates, microfluidic devices, cartridges, DLD devices, etc.) and methods for purifying particles or cells, which may comprise chimeric antigen receptor (CAR) T and NK cells. The microfluidic cartridges (i.e. devices, chips, cassettes, plates, microfluidic devices, cartridges, DLD devices, etc.) may be any of those described herein. The use of the described cartridges may allow for production of more highly effective CAR T or NK cells by providing a purer T or NK cell product for downstream genetic engineering and CAR T or NK cell production. A more effective CAR T or NK cell may be produced by removing platelets that other methods for producing CAR T or NK cells cannot accomplish.

A method for producing chimeric antigen receptor (CAR) T or NK cells may comprise obtaining sample comprising T or NK cells and separating the T or NK cells from contaminants. Contaminants may comprise platelets, or other contaminants described herein. Separating contaminants may comprise applying the sample to the one or more sample inlets of any of the cartridges or devices described herein., flowing the sample to the outlets of the cartridge, obtaining a product enriched in T or NK cells from the product outlet, and genetically engineering the T cells in the enriched product to product chimeric antigen receptors on the surface of the T NK cells. The sample of the method may include an apheresis product or a leukapheresis product. The genetically engineering of the method may comprise genetic engineering methods as described herein. The method may further comprise expanding the CAR T or NK cells by growing the cell in vitro.

Some commercial examples of CAR T cell therapeutics that can be engineered according to the device and methods herein include axicabtagene ciloleucel, tisagenlecleucel, and brexucabtagene autoleucel.

IV. Separation Processes that Use DLD

The DLD devices described herein can be used to purify cells, cellular fragments, cell adducts, or nucleic acids. Separation and purification of blood components using devices can be found, for example, in US Publication No. US 2016/0139012, the teaching of which is incorporated by reference herein in its entirety.

The purity, yields and viability of cells produced by DLD methods will vary based on a number of factors including the nature of the starting material, the exact procedure employed and the characteristics of the DLD device. Preferably, purifications, yields and viabilities of at least 60% should be obtained with, higher percentages, at least 70, 80 or 90% being more preferred.

In an aspect, the present disclosure provides methods for enriching target particles or target cells of a predetermined size from contaminants in a sample. Methods for enriching target particles or target cells use any cartridge, microfluidic cartridge, cassette, chip, device, fluidic device, or microfluidic device as described elsewhere herein. A method may comprise obtaining a sample comprising target particles or target cells and the contaminants. The method may further comprise separating the target particles or target cells from the contaminants by applying the sample to one or more sample inlets on any of the cartridges, cassettes, or devices described herein. The method may further comprise flowing the sample to the outlets on any of the cartridges, cassettes, or devices described herein. The method may further comprise obtaining a product enriched in target particles or target cells from one or more outlets while removing the contaminants. The method may result in a superior ability to purify or separate cells or particles from contaminants, creating greater cells yields, improved ability to expand the product in vitro, and an enriched cell product more amenable to transduction or other genetic engineering.

The method may entail the used of deterministic lateral displacement whereby the device has a critical size as described herein and the contaminants and the target particles or target cells are separated on the basis of having different critical size. The method may comprise flowing a sample containing the target particles or target cells and contaminants to any of the of the cartridges, cassettes, or devices described herein, wherein the target particles or target cells have a size larger than a critical size of the array of obstacles and at least some contaminants have sizes smaller than the critical size of the array of obstacles and wherein target cells or target particles flow to the one or more product outlets where a product enriched in target cells or target particles is obtained and contaminants with a size smaller than the critical size of the array of obstacles flow to one more waste outlets. The method may comprise flowing a sample containing the target particles or target cells and contaminants to any of the of the cartridges, cassettes, or devices described herein, wherein the target particles or target cells have a size smaller than a critical size of the array of obstacles and at least some contaminants have sizes larger than the critical size of the array of obstacles and wherein target cells or target particles flow to the one or more product outlets where a product enriched in target cells or target particles is obtained and contaminants with a size larger than the critical size of the array of obstacles flow to one more waste outlets.

The method may comprise flowing a sample containing the target particles or target cells and contaminants to any of the of the cartridges, cassettes, or devices described herein, at a constant flow rate or a variable flow rate. The cartridge flow rate of the method may be about 400 mL per hour. The cartridge flow rate of the method may be about 100 mL per hour to about 1,000 mL per hour. The cartridge flow rate of the method may be about 100 mL per hour to about 200 mL per hour, about 100 mL per hour to about 400 mL per hour, about 100 mL per hour to about 800 mL per hour, about 100 mL per hour to about 1,000 mL per hour, about 200 mL per hour to about 400 mL per hour, about 200 mL per hour to about 800 mL per hour, about 200 mL per hour to about 1,000 mL per hour, about 400 mL per hour to about 800 mL per hour, about 400 mL per hour to about 1,000 mL per hour, or about 800 mL per hour to about 1,000 mL per hour. The cartridge flow rate of the method may be about 100 mL per hour, about 200 mL per hour, about 400 mL per hour, about 800 mL per hour, or about 1,000 mL per hour.

The cartridge flow rate of the method may be at least about 100 mL per hour, about 200 mL per hour, about 400 mL per hour, or about 800 mL per hour. The cartridge flow rate of the method may be at most about 200 mL per hour, about 400 mL per hour, about 800 mL per hour, or about 1,000 mL per hour.

The method may comprise an internal pressure within the cartridge. The internal pressure of the cartridge may be at least about 15 pounds per square inch. The internal pressure of the cartridge may be at least about 1.5 pounds per square inch to about 50 pounds per square inch. The internal pressure of the cartridge may be at least about 1.5 pounds per square inch to about 5 pounds per square inch, about 1.5 pounds per square inch to about 10 pounds per square inch, about 1.5 pounds per square inch to about 15 pounds per square inch, about 1.5 pounds per square inch to about 20 pounds per square inch, about 1.5 pounds per square inch to about 50 pounds per square inch, about 5 pounds per square inch to about 10 pounds per square inch, about 5 pounds per square inch to about 15 pounds per square inch, about 5 pounds per square inch to about 20 pounds per square inch, about 5 pounds per square inch to about 50 pounds per square inch, about 10 pounds per square inch to about 15 pounds per square inch, about 10 pounds per square inch to about 20 pounds per square inch, about 10 pounds per square inch to about 50 pounds per square inch, about 15 pounds per square inch to about 20 pounds per square inch, about 15 pounds per square inch to about 50 pounds per square inch, or about 20 pounds per square inch to about 50 pounds per square inch. The internal pressure of the cartridge may be at least about 1.5 pounds per square inch, about 5 pounds per square inch, about 10 pounds per square inch, about 15 pounds per square inch, about 20 pounds per square inch, or about 50 pounds per square inch. The internal pressure of the cartridge may be at least about 1.5 pounds per square inch, about 5 pounds per square inch, about 10 pounds per square inch, about 15 pounds per square inch, or about 20 pounds per square inch. The internal pressure of the cartridge may be at least at most about 5 pounds per square inch, about 10 pounds per square inch, about 15 pounds per square inch, about 20 pounds per square inch, or about 50 pounds per square inch.

The target particles or target cells of the method may comprise stem cells, thrombocytes, synoviocytes, fibroblasts, beta cells, liver cells, megakaryocytes, pancreatic cells, DE3 lysogenized cell, yeast cells, plant cells, algae cells, monocytes, T cells, B cells, regulatory T cells, macrophages, dendritic cells, granulocytes, innate lymphoid cells, natural killer cells, leukocytes, peripheral blood mononuclear cells, CD3+ cells, neurons, platelets, cancer cells, muscle cells, or epithelial cells. The method may comprise enriching target particles or target cells to produce enriched target cells comprising stem cells, thrombocytes, synoviocytes, fibroblasts, beta cells, liver cells, megakaryocytes, pancreatic cells, DE3 lysogenized cell, yeast cells, plant cells, algae cells, monocytes, T cells, B cells, regulatory T cells, macrophages, dendritic cells, granulocytes, innate lymphoid cells, natural killer cells, leukocytes, peripheral blood mononuclear cells, CD3+ cells, neurons, platelets, cancer cells, muscle cells, or epithelial cells. The contaminants of the method may comprise stem cells, thrombocytes, synoviocytes, fibroblasts, beta cells, liver cells, megakaryocytes, pancreatic cells, DE3 lysogenized cell, yeast cells, plant cells, algae cells, monocytes, T cells, B cells, regulatory T cells, macrophages, dendritic cells, granulocytes, innate lymphoid cells, natural killer cells, leukocytes, peripheral blood mononuclear cells, CD3+ cells, neurons, platelets, cancer cells, muscle cells, or epithelial cells. For example, the target cells may be peripheral blood mononuclear cells and the contaminants may be platelets. For example, the target cells may be CD3+ cells and the contaminants may be platelets. The method may result in the removal of more than 90% of the platelets. The method may result in the removal of about 50% of the platelets to about 99% of the platelets. The method may result in the removal of about 50% of the platelets to about 75% of the platelets, about 50% of the platelets to about 80% of the platelets, about 50% of the platelets to about 90% of the platelets, about 50% of the platelets to about 95% of the platelets, about 50% of the platelets to about 99% of the platelets, about 75% of the platelets to about 80% of the platelets, about 75% of the platelets to about 90% of the platelets, about 75% of the platelets to about 95% of the platelets, about 75% of the platelets to about 99% of the platelets, about 80% of the platelets to about 90% of the platelets, about 80% of the platelets to about 95% of the platelets, about 80% of the platelets to about 99% of the platelets, about 90% of the platelets to about 95% of the platelets, about 90% of the platelets to about 99% of the platelets, or about 95% of the platelets to about 99% of the platelets. The method may result in the removal of about 50% of the platelets, about 75% of the platelets, about 80% of the platelets, about 90% of the platelets, about 95% of the platelets, or about 99% of the platelets. The method may result in the removal of at least about 50% of the platelets, about 75% of the platelets, about 80% of the platelets, about 90% of the platelets, or about 95% of the platelets. The method may result in the removal of at most about 75% of the platelets, about 80% of the platelets, about 90% of the platelets, about 95% of the platelets, or about 99% of the platelets.

The method may comprise modifying the enriched target cells. The method may comprise genetically engineering the enriched target cells to obtain genetically engineered target cells. Genetically engineering includes transfecting or transducing the target cells with a recombinant nucleic acid. Methods of genetic engineering may include the use of TALENs, Zinc Finger Nucleases, CRISPR-Cas associated proteins, homologous recombination, viral vectors, or heterologous plasmids. The method may also include expanding the enriched target cells or genetically engineered cells by culturing them in vitro.

### V. Technological Background

“Obstacle array” devices have been described, and their basic operation is explained, for example in U.S. Pat. No. 7,150,812, which is incorporated herein by reference in its entirety. A bump array operates essentially by segregating particles passing through an array (generally, a periodically-ordered array) of obstacles, with segregation occurring between particles that follow the direction of bulk fluid flow and particles that follow an “array direction” that is offset from the direction of bulk fluid flow.

### A. Fractionation Range

Objects separated by size on microfluidic devices include cells, biomolecules, inorganic beads, and other objects. Typical sizes fractionated range from 100 nanometers to 50 micrometers. However, larger and smaller particles may also sometimes be fractionated.

### B. Volumes

Depending on the design of a device or combination of devices, the rate at which a sample can be processed will vary considerably. Preferably devices and assemblies should be able to process greater than 500 ml of sample in an hour.

### C. Channels

A device can comprise one or multiple channels with one or more inlets and one or more outlets. Inlets may be used for sample or crude (i.e., unpurified) fluid compositions, for buffers or to introduce reagents. Outlets may be used for collecting product or may be used as an outlet for waste. Channels may be about 0.5 to 100 mm in width and about 2-200 mm long but different widths and lengths are also possible. Depth may be 1-1000 μm and there may be anywhere from 1 to 500 channels or more present on a device.

Specific embodiments of the various aspects described herein can be illustrated by the following numbered embodiments.

1. A microfluidic device for purifying target particles or target cells of a predetermined size from contaminants in a sample, the device comprising a planar support having a top surface and a bottom surface, wherein the top and/or bottom surface comprises at least one embedded channel extending from one or more sample inlets and one or more distinct fluid inlets, to one or more product outlets and one or more distinct waste outlets; wherein: (a) when fluid is applied to a channel through a sample and/or fluid inlet, it flows through the channel toward the outlets, thereby defining a direction of bulk fluid flow; (b) the channel comprises an array of obstacles arranged in columns extending longitudinally along the channel, and rows extending laterally across the channel, wherein the obstacles are positioned so as to define a critical size such that, when a sample is applied to an inlet of the device and flows to an outlet, particles or cells in the sample larger than the critical size are separated from particles or cells in the sample smaller than the critical size; and wherein: (i) adjacent obstacles in a row are separated by a gap, G1, that is perpendicular to the direction of bulk fluid flow; (ii) adjacent obstacles in a column are separated by a gap, G2, which is parallel to the direction of bulk fluid flow; (iii) the ratio of the size of gap G2 to the size of gap G1 does not equal 1; (iv) each subsequent row of obstacles is shifted laterally with respect to the previous row, thereby defining an array direction that deviates from the direction of bulk fluid flow by a tilt angle (c); (v) obstacles have at least two vertices such that each gap is flanked on either side by at least one vertex. 2. The device of embodiment 1, further comprising an obstacle bonding layer that is bonded to a surface of the planar support and bonded to obstacles in channels embedded in the surface to prevent fluid or sample from flowing over obstacles during operation of the device. 3. The microfluidic device of embodiment 2, wherein the obstacle bonding layer comprises one or more passages fluidically connected to the sample inlets of the channels which permit the flow of sample into the channels and one or more passages fluidically connected to the outlets of the channels that permit the flow of fluid out from the outlets. 4. The microfluidic device of any one of embodiments 1-3, wherein the target particles or target cells have a size larger than the critical size of the device and at least some contaminants have sizes smaller than the critical size and wherein obstacles are disposed in a manner such that, when said sample is applied to an inlet of the device and fluidically passed through the channel, target cells or target particles flow to the one or more product outlets where an enriched product comprising target cells or target particles is obtained and contaminants with a size smaller than the critical size flow to one more waste outlets. 5. The microfluidic device any one of embodiments 1-4, wherein obstacles have a polygonal shape. 6. The microfluidic device of embodiment 5, wherein obstacles have a diamond or hexagonal shape. 7. The microfluidic device of embodiment 5 or embodiment 6, wherein obstacles are elongated perpendicularly to the direction of bulk fluid such that they have a horizontal length (P1) that is different from their vertical length (P2). 8. The microfluidic device of embodiment 6, wherein P1 is a least 15% longer than P2. 9. The microfluidic device of embodiment 6, wherein P1 is 10-150% longer than P2. 10. The microfluidic device of embodiment 6, wherein P1 is 15-100% longer than P2. 11. The microfluidic device of embodiment 6, wherein P1 is 20-70% longer than P2. 12. The microfluidic device of any one of embodiments 1-11, wherein obstacles have vertices that extend into parallel gaps such that the gaps are flanked on either side by one or more vertices pointing toward one another but not directly opposite one another. 13. The microfluidic device of any one of embodiments 1-12, wherein obstacles have vertices that extend into perpendicular gaps such that the gaps are flanked on either side by vertices pointing toward one another and that are directly opposite one another. 14. The microfluidic device of any one of embodiments 1-13, wherein the sample inlet or inlets are separated from fluid inlet or inlets by a separator wall that extends from the sample inlet or inlets into the array of obstacles in the channel toward the outlets and that is oriented parallel to the direction of bulk fluid flow. 15. The microfluidic device of embodiment 14, wherein the separator wall extends for at least 10% of the length of the array of obstacles. 16 The microfluidic device of embodiment 14, wherein the separator wall extends for at least 20% of the length array of obstacles. 17. The microfluidic device of embodiment 14, wherein the separator wall extends for at least 40% of the length array of obstacles. 18. The microfluidic device of embodiment 14, wherein the separator wall extends for at least 60% of the length array of obstacles. 19. The microfluidic device of any one of embodiments 1-18, wherein the inlets and/or outlets of the device are connected to a peristaltic pump. 20. A stacked separation assembly comprising at least two of the microfluidic devices of any one of embodiments 1-19. 21. A stacked separation assembly comprising a first microfluidic device selected from the microfluidic devices of any one of embodiments 1-19, and one or more stacked microfluidic devices also selected from the microfluidic devices of any one of embodiments 1-19, wherein: (a) the bottom surface of each stacked device is in contact with either the top surface or an obstacle bonding layer on the top surface of the first microfluidic device, or with the top surface or the obstacle bonding layer on the top surface of another stacked device; (b) sample is provided to the sample inlets though a first common manifold; (c) fluid is supplied to fluid inlets through a second manifold that may or may not be the same as the first manifold; (d) product is removed from the product outlets through one or more conduits; (e) waste is removed from the waste outlets through one or more conduits that are different from the one or more conduits of (d); (f) the first microfluidic device and the stacked microfluidic devices are optionally mounted inside a common outer casing. 22. The stacked separation assembly of embodiment 22, wherein the assembly comprises at least 2 stacked microfluidic devices. 23. The stacked separation assembly of embodiment 22, further comprising at least one reservoir bonding layer which is attached to the bottom surface of the first microfluidic device and/or to the top surface of a stacked microfluidic device and which, at a first end, comprises one or more passages permitting the flow of fluid from to inlets on the channels and at a second end, opposite from the first end, one or more passages that permit the flow of fluid from the product and waste outlets of channels, and wherein the passages at the first and second ends of said reservoir layer are separated by material impermeable to fluid. 24. The stacked separation assembly of any one of embodiment 22 or 23, wherein both the top and bottom surfaces of the planar support of the one or more microfluidic devices comprise one or more the channels with obstacles for separating target particles or target cells. 25. A method for purifying target particles or target cells of a predetermined size from contaminants in a sample, the method comprising: (a) obtaining a sample comprising said target particles or target cells and said contaminants; (b) separating the target particles or target cells from the contaminants by: (i) applying the sample to one or more sample inlets on the microfluidic device of any one of embodiments 1-21 or on the first microfluidic device or a stacked device of any one of embodiments 22-24; (ii) flowing the sample to the outlets on the device of any one of embodiments 1-21 or on the first microfluidic device or a stacked device of any one of embodiments 22-24; and (iii) obtaining a product enriched in target particles or target cells from one or more or outlets. 26. The method of embodiment 25, wherein the target particles or target cells have a size larger than the critical size of the array of obstacles and at least some contaminants have sizes smaller than the critical size and wherein target cells or target particles flow to the one or more product outlets where a product enriched in target cells or target particles is obtained and contaminants with a size smaller than the critical size flow to one more waste outlets. 27. The method of embodiment 26, wherein the sample is blood or is derived from blood. 28. The method of embodiment 26, wherein the sample is an apheresis or leukapheresis sample. 29. The method of embodiment 27 or 28, wherein the sample comprises platelets as contaminants. 30. The method of embodiment 29, wherein the method results in the removal of at least 80% of the platelets from the sample. 31. The method of embodiment 29, wherein the method results in the removal of at least 90% of the platelets from the sample. 32 The method of embodiment 29, wherein the method results in the removal of at least 95% of the platelets from the sample. 33 The method of any one of embodiments 27-31, wherein the target cells are leukocytes. 34. The method of any one of embodiments 27-31, wherein the target cells are stem cells. 35. The method of any one of embodiments 27-31, wherein the target cells are B-cells, T cells, NK-cells, monocytes or progenitor cells. 36. The method of any one of embodiments 27-31, wherein the target cells are dendritic cells. 37. The method of any one of embodiments 25-36, wherein the sample is obtained from a patient. 38. The method of embodiment 37, wherein the patient has cancer, an autoimmune disease or an infectious disease. 39. The method of any one of embodiments 25-38, further comprising genetically engineering the purified target cells. 40. The method of embodiment 39, wherein said genetic engineering comprises transfecting or transducing the target cells with a recombinant nucleic acid. 41. The method of embodiment 39 or 40, wherein the genetically engineered target cells are expanded by culturing them in vitro. 42. A method of producing chimeric antigen receptor (CAR) T cells, comprising: (a) obtaining a sample comprising T cells; (b) separating the T cells from contaminants by: (i) applying the sample to one or more sample inlets on the microfluidic device of any one of embodiments 1-21 or on the first microfluidic device or a stacked device of any one of embodiments 22-24; (ii) flowing the sample to the outlets of the device; and (iii) obtaining a product enriched in T cells from the product outlet; (c) genetically engineering the T cells in the enriched product obtained in step b) to produce the chimeric antigen receptors (CARs) on their surface. 43. The method of embodiment 42, wherein sample is blood, an apheresis product or a leukapheresis product from a patient. 44. The method of either embodiment 42 or 43, wherein said genetic engineering comprises transfecting or transducing the target cells and the genetically engineered target cells are expanded further by growing the cells in vitro. 45. The method of any one of embodiments 42-44, wherein separation is accomplished by performing deterministic lateral displacement on the microfluidic device. 46. The method of any one of embodiments 42-44, wherein said sample is obtained from a patient with cancer, an autoimmune disease or an infectious disease. 47. The method of embodiment 46 wherein, after obtaining the sample, the T cells are bound to one or more carriers in a way that promotes DLD separation. 48. CAR T cells made by the method of any one of embodiments 42-47.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof

What is claimed is:

1. A microfluidic cartridge for purifying target particles or target cells of a predetermined size from contaminants in a sample, the cartridge comprising a first and a second planar support the first and second planar support each having a top surface and a bottom surface, wherein the top surface of the first and/or second planar support comprises at least one embedded channel extending from one or more inlets to one or more outlets; the at least one embedded channel comprising a plurality of obstacles, wherein the microfluidic cartridge comprises at least one void space configured to be deformed when assembling the first and second planar supports into the microfluidic cartridge.

2. The microfluidic cartridge of claim 1, wherein the bottom surface of the first and second planar support comprise at least one void space configured to be deformed when the bottom of the first planar support is pressed to the bottom of the second planar support.

3. The microfluidic cartridge of claim 1, wherein the at least one void space is configured to prevent damage, displacement, or deformation of the at least one embedded channel, the one or more inlets, the one or more outlets, the plurality of obstacles, or a combination thereof.

4. The microfluidic cartridge of any one of claims 1 to 3, wherein the at least one void space is configured to prevent damage, displacement, or deformation of the plurality of obstacles.

5. The microfluidic cartridge of any one of claims 1 to 3, comprising a 1:1 ratio of void spaces to channels.

6. The microfluidic cartridge of any one of claims 1 to 3, wherein the at least one void space comprises a total surface area that is at least about 90% of a total surface area of the at least one embedded of channel.

7. The microfluidic cartridge of any one of claims 1 to 3, wherein the at least one void space comprises a total surface area that is at least about 100% of a total surface area of the at least one embedded channel.

8. The microfluidic cartridge of any one of claims 1 to 3, wherein the at least one void space comprises a total surface area that is at least about 110% of a total surface area of the at least one embedded channel.

9. The microfluidic cartridge of any one of claims 1 to 8, wherein the at least one void space is separated into two or more void spaces positioned on the bottom surface of the first and/or second planar support opposite the array of obstacles.

10. The microfluidic cartridge of any one of claims 1 to 9, wherein the planar support is fabricated from two layers of material bonded together.

11. The microfluidic cartridge of any one of claims 1 to 10, further comprising an obstacle bonding layer that is bonded to a surface of the planar support and bonded to a top surface of the plurality of obstacles in the at least one embedded channel to prevent fluid or sample from flowing over the plurality of obstacles during operation of the cartridge.

12. The microfluidic cartridge of claim 11, wherein the obstacle bonding layer comprises one or more passages fluidically connected to the one or more inlets of the at least one embedded channel which permits the flow of sample into the at least one embedded channel and one or more passages fluidically connected to the one or more outlets of the at least one embedded channel that permits the flow of fluid out from the one or more outlets.

13. The microfluidic cartridge of any one of claims 1 to 12, wherein the obstacles are positioned so as to define a critical size of the cartridge such that, when a sample is applied to an inlet of the cartridge and flows to an outlet, particles or cells in the sample larger than the critical size are separated from particles or cells in the sample smaller than the critical size.

14. The microfluidic cartridge of claim 13, wherein the one or more outlets comprise at least one product outlet, wherein the target particles or target cells that have a size larger than the critical size of the cartridge are directed to the at least one product outlet.

15. The microfluidic cartridge of claim 13, wherein the one or more outlets comprise at least one waste outlet, and the contaminants that have a size smaller than the critical size of the cartridge flow to the at least one waste outlet.

16. The microfluidic cartridge of any one of claims 1 to 15, wherein the plurality of obstacles have a diamond shape.

17. The microfluidic cartridge of any one of claims 1 to 15, wherein the plurality of obstacles have a circular or ellipsoid shape.

18. The microfluidic cartridge of any one of claims 1 to 15, wherein the plurality of obstacles have a hexagonal shape.

19. The microfluidic cartridge of claims 16 to 18, wherein the plurality of obstacles are elongated perpendicularly to the direction of fluid flow such that they have a horizontal length (P1) that is different from their vertical length (P2).

20. The microfluidic cartridge of claim 19, wherein P1 is about 10 μm to about 160 μm and P2 is about 5 μm to about 80 μm.

21. The microfluidic cartridge of claim 19, wherein P1 is about 10 μm to about 80 μm and P2 is about 15 μm to about 60 μm.

22. The microfluidic cartridge of claim 19, wherein P1 is about 15 μm to about 30 μm and P2 is about 25 μm to about 45 μm.

23. The microfluidic cartridge of claim 19, wherein P1 is about 40 μm and P2 is about 20 μm.

24. The microfluidic cartridge of claim 19, wherein P1 is 50 to 150% longer than P2.

25. The microfluidic cartridge of any one of claims 1 to 24, wherein the plurality of obstacles have vertices that extend into parallel gaps such that the gaps are flanked on either side by one or more vertices pointing toward one another but not directly opposite one another.

26. The microfluidic cartridge of any one of claims 1 to 24, wherein the plurality of obstacles have vertices that extend into perpendicular gaps such that the gaps are flanked on either side by vertices pointing toward one another and that are directly opposite one another.

27. The microfluidic cartridge of any one of claims 1 to 26, wherein the plurality of obstacles is arranged into at least at least 1 column.

28. The microfluidic cartridge of any one of claims 1 to 26, wherein the plurality of obstacles is arranged into at least at least 10 columns.

29. The microfluidic cartridge of any one of claims 1 to 26, wherein the plurality of obstacles is arranged into at least at least 30 columns.

30. The microfluidic cartridge of any one of claims 1 to 26, wherein the plurality of obstacles is arranged into at least 50 columns.

31. The microfluidic cartridge of any one of claims 1 to 26, wherein the plurality of obstacles is arranged into at least about 60 columns.

32. The microfluidic cartridge of any one of claims 1 to 31, wherein the plurality of obstacles is arranged into at least at least about 50 rows.

33. The microfluidic cartridge of any one of claims 1 to 31, wherein the plurality of obstacles is arranged into at least at least about 100 rows.

34. The microfluidic cartridge of any one of claims 1 to 31, wherein the plurality of obstacles is arranged into at least at least about 300 rows.

35. The microfluidic cartridge of any one of claims 1 to 31, wherein the plurality of obstacles is arranged into at least at least about 600 rows.

36. The microfluidic cartridge of any one of claims 1 to 35, wherein the first or second planar support comprise at least 10 embedded channels.

37. The microfluidic cartridge of any one of claims 1 to 35, wherein the first and/or second planar support comprise at least 20 embedded channels.

38. The microfluidic cartridge of any one of claims 1 to 35, wherein the first and/or second planar support comprise about 28 embedded channels.

39. The microfluidic cartridge of any one of claims 1 to 35, wherein the first and/or second planar support comprise about 30 embedded channels.

40. The microfluidic cartridge of any one of claims 1 to 35, wherein the first and/or second planar support comprise at least about 50 embedded channels.

41. The microfluidic cartridge of any one of claims 1 to 40, wherein the one or more inlets are comprised of at least one or more sample inlets and at least one or more fluid inlets; wherein the at least one or more sample inlets are separated from the at least one or more fluid inlets by a separator wall that extends from the one or more sample inlets into the array of obstacles in the at least one embedded channel toward the outlets and that is oriented parallel to the direction of fluid flow.

42. The microfluidic cartridge of claim 41, wherein the separator wall extends for at least 10% of the length of the plurality of obstacles.

43. The microfluidic cartridge of claim 41, wherein the separator wall extends for at least 20% of the length plurality of obstacles.

44. The microfluidic cartridge of claim 41, wherein the separator wall extends for at least 60% of the length plurality of obstacles.

45. The microfluidic cartridge of any one of claims 1 to 44, wherein the one or more inlets, the one or more outlets, or both, are fluidically connected to a first peristaltic pump, a second peristaltic pump, or both.

46. The microfluidic cartridge of claim 45, wherein the first peristaltic pump and the second peristaltic pump are fluidically connected in serial.

47. The microfluidic cartridge of claim 45, wherein the first peristaltic pump and the second peristaltic pump are fluidically connected in parallel.

48. The microfluidic cartridge of any one of claims 1 to 47, wherein the cartridge is fabricated from a polymer.

49. The microfluidic cartridge of claim 48, wherein the polymer is a thermoplastic polymer.

50. The microfluidic cartridge of claim 48, wherein the thermoplastic polymer is chosen from the group comprising of high-density polyethylene, polypropylene, polyethylene terephthalate, polycarbonate, or cyclic olefin copolymer.

51. The microfluidic cartridge of claim 48, wherein the thermoplastic polymer is cyclic olefin copolymer.

52. A microfluidic assembly comprising a plurality of microfluidic cartridges of any one of claims 1 to 51, wherein the plurality of microfluidic cartridges are in fluid connection.

53. The microfluidic assembly of claim 52, wherein the microfluidic cartridges are stacked.

54. The microfluidic assembly of claim 52, wherein the plurality of microfluidic cartridges is two.

55. The microfluidic assembly of claim 52, wherein the microfluidic cartridges are in fluid connection in parallel.

56. The microfluidic assembly of claim 52, wherein the microfluidic cartridges are in fluid connection in series.

57. A method of manufacturing the microfluidic cartridge of any one of claims 1 to 56, wherein the cartridge is fabricated by pressing the bottoms of the first and the second planar support together such that the array of obstacles are not deformed.

58. The method of manufacturing of claim 57, wherein the at least one embedded channel, obstacles, or both are fabricated by embossing, hot embossing, roll to roll embossing, or injection molding.

59. The method of manufacturing of any one of claim 57 or 58, wherein the microfluidic cartridge is UV-light cured during fabrication.

60. A method for enriching target particles or target cells of a predetermined size from contaminants in a sample, the method comprising:
a) obtaining a sample comprising the target particles or target cells and the contaminants;
b) separating the target particles or target cells from the contaminants by:
i) applying the sample to one or more sample inlets on the microfluidic cartridge of any one of claims 1 to 56;
ii) flowing the sample to the outlets on the cartridge of any one of claims 1 to 56; and
iii) obtaining a product enriched in target particles or target cells from one or more or outlets while removing the contaminants.

61. The method of claim 60, wherein the target particles or target cells have a size larger than a critical size of the array of obstacles and at least some contaminants have sizes smaller than the critical size of the array of obstacles and wherein target cells or target particles flow to the one or more product outlets where a product enriched in target cells or target particles is obtained and contaminants with a size smaller than the critical size of the array of obstacles flow to one more waste outlets.

62. The method of claim 60 or 61, wherein the flow rate of the cartridge is about 400 mL per hour.

63. The method of claim 60 or 61, wherein the flow rate of the cartridge is at least about 100 mL per hour or greater.

64. The method of claim 60 or 61, wherein the flow rate of the cartridge is at least about 300 mL per hour or greater.

65. The method of claim 60 or 61, wherein the flow rate of the cartridge is about 1000 mL per hour.

66. The method of claim 60 or 61, wherein the internal pressure of the cartridge is at least about 1.5 pounds per square inch or greater.

67. The method of claim 60 or 61, wherein the internal pressure of the cartridge is about 15 pounds per square inch.

68. The method of claim 60 or 61, wherein the internal pressure of the cartridge is about 50 pounds per square inch or less.

69. The method of claim 60 or 61, wherein the internal pressure of the cartridge is from about 10 pounds per square inch to about 20 pounds per square inch.

70. The method of any one of claims 60 to 69, wherein the sample is blood or a blood related product.

71. The method of any one of claims 60 to 69, wherein the sample is an apheresis or leukapheresis sample.

72. The method of any one of claims 60 to 71, wherein the sample comprises platelets as contaminants.

73. The method of claim 72, wherein the method results in the removal of at least 80% of the platelets from the sample.

74. The method of claim 72, wherein the method results in the removal of at least 90% of the platelets from the sample.

75. The method of claim 72, wherein the method results in the removal of at least 95% of the platelets from the sample.

76. The method of any one of claims 60 to 75, wherein the enriched target cells comprise leukocytes.

77. The method of any one of claims 60 to 75, wherein the enriched target cells comprise stem cells.

78. The method of any one of claims 60 to 75, wherein the enriched target cells comprise peripheral blood mononuclear cells.

79. The method of claim 78, wherein the peripheral blood mononuclear cells comprise CD3+ cells.

80. The method of any one of claims 60 to 79, further comprising genetically engineering the enriched target cells, to obtain genetically engineered target cells.

81. The method of claim 80, wherein said genetic engineering comprises transfecting or transducing the target cells with a recombinant nucleic acid.

82. The method of claim 80 or 81, wherein the enriched target cells or genetically engineered target cells are expanded by culturing them in vitro.

83. A method of producing chimeric antigen receptor (CAR) T cells, comprising:
a) obtaining a sample comprising T cells;
b) separating the T cells from contaminants by:
i) applying the sample to one or more sample inlets on the microfluidic cartridge of any one of claims 1 to 56;
ii) flowing the sample to the outlets of the cartridge; and
iii) obtaining a product enriched in T cells from the product outlet;
c) genetically engineering the T cells in the enriched product obtained in step b) to produce the chimeric antigen receptors (CARs) on their surface.

84. The method of claim 83, wherein the sample is blood, an apheresis product or a leukapheresis product.

85. The method of claim 83 or 84, wherein said genetically engineering the T cells comprises transfecting or transducing the target cells and the genetically engineered target cells are expanded further by growing the cells in vitro.

86. A method of producing chimeric antigen receptor (CAR) natural killer cells, comprising:
a) obtaining a sample comprising natural killer cells;
b) separating the natural killer cells from contaminants by:
i) applying the sample to one or more sample inlets on the microfluidic cartridge of any one of claims 1 to 56;
ii) flowing the sample to the outlets of the cartridge; and
iii) obtaining a product enriched in natural killer cells from the product outlet;
c) genetically engineering the natural killer cells in the enriched product obtained in step b) to produce the chimeric antigen receptors (CARs) on their surface.

87. The method of claim 86, wherein the sample is a blood sample, an apheresis product, or a leukapheresis product.

88. The method of claim 86 or 87, wherein said genetically engineering the natural killer cells comprises transfecting or transducing the target cells and the genetically engineered target cells are expanded further by growing the cells in vitro.
